(19) United States
(12) Patent Application Publication
(10) Pub. No.: US 2024/0197977 A1
(43) Pub. Date: Jun. 20, 2024

(54) IMPROVED IMMUNE RESPONSE AND DECREASED IMMUNOPARALYSIS WITH IMMUNOMODULATING TREATMENT

(71) Applicant: CYTOSORBENTS, INC., Princeton, NJ (US)

(72) Inventors: Phillip P CHAN, Princeton, NJ (US); Vincent J. CAPPONI, Princeton, NJ (US); Thomas D. Golobish, Princeton, NJ (US); Wei-Tai YOUNG, Princeton, NJ (US); Ophir ORTIZ, Princeton, NJ (US); Ritu TRIPATHI, Princeton, NJ (US)

(21) Appl. No.: 17/919,053

(22) PCT Filed: Apr. 14, 2021
(86) PCT No.: PCT/US2021/027369

§ 371 Date: Sep. 13, 2023

Related U.S. Application Data
(60) Provisional application No. 63/009,515, filed on Apr. 14, 2020.

(51) Int. Cl. A61M 1/36; B01J 20/26; B01J 20/28

(52) U.S. Cl. CPC A61M 1/3679; B01J 20/267; B01J 20/28073; B01J 20/28076; B01J 20/28083; B01J 20/28085

(57) ABSTRACT

The invention concerns methods of treating immunoparalysis by the attenuation via removal of inflammatory mediators and/or checkpoint inhibitor proteins in a subject, comprising treating a subject with a therapeutically effective amount of porous biocompatible polymer sorbent comprising a range of pore diameters between about 50 Å to about 3000 Å and a pore volume between about 0.5 cc/g to about 3.0 cc/g dry polymer; or comprising a range of pore diameters between about 50 Å to about 40,000 Å and a pore volume between about 0.5 cc/g to about 5.0 cc/g dry polymer.

### CROSS-REFERENCE TO RELATED APPLICATIONS

This application is related to U.S. Patent Application No. 63/009,515, filed Apr. 14, 2020, the entire disclosure of which is incorporated herein in its entirety.

### GOVERNMENT SUPPORT

One or more of the inventions disclosed herein was made with government support under Contract No. N66001-12-C-4199 awarded by the Defense Advanced Research Projects Agency and SSC Pacific. The government may have certain rights in the invention.

### TECHNICAL FIELD

The invention concerns immunomodulating therapies for the treatment of conditions or critical injuries that are affected by excessive levels of inflammatory or anti-inflammatory mediators and/or immune checkpoint proteins, leading to an immunosuppressive state called immunoparalysis. Anergy is a synonym to immunoparalysis.

### BACKGROUND

A plethora of diseases, acute and chronic conditions, syndromes, and critical injuries can produce a dysregulated immune response, leading to multiple organ failure, infection, compromised healing, and increased mortality. Immunoparalysis may play a critical role in this progression.

Sepsis is one example of a syndrome with high mortality rates for which there is no clinical solution. In the U.S. alone, approximately 1.7 million patients each year develop sepsis, and nearly 270,000 patients succumb to multiple organ failure and death (https://www.cdc.gov/features/get-ahead-sepsis/index.html). Bacteria, virus, or fungi can cause sepsis. The most common cause of sepsis is bacteria, and the most common presentation is pneumonia (https://www.nigms.nih.gov/education/Documents/Sepsis.pdf). Although this syndrome has been investigated for decades, treatment options remain limited to supportive care. These include antibiotics, vasopressors, mechanical ventilation (in cases of acute respiratory syndrome secondary to sepsis), and fluid therapy. Unfortunately, none of these options treat the underlying dysregulated inflammatory response, nor do they address the immunoparalysis that occurs during and following sepsis. Immunoparalysis, which starts at the beginning of the proinflammatory phase and can remain even after systemic inflammatory release syndrome (SIRS) phase has subsided, is now recognized as a leading cause of death in sepsis and septic shock patients (van Ton, Annemieke M. Peters, et al. “Precision immunotherapy for sepsis.” Frontiers in immunology 9 (2018)). A loss of immune system function can lead to significantly compromised healing and recovery, an increased risk of infection, abnormal tissue remodeling, and many other complications.

The deleterious effects of sepsis on the immune system are significant and are known to affect both the innate and adaptive response. Leukocyte trafficking and function is impaired, thereby rendering the body highly susceptible to nosocomial infections and low virulence pathogens. Under septic conditions, activated leukocytes adhere to activated endothelium tissue and produce proteolytic enzymes and reactive oxygen species for pathogen clearance. As sepsis progresses, this response becomes dysregulated, causing impaired chemotaxis of activated leukocytes throughout the body irrespective of infection site, producing tissue damage and subsequent multiple organ failure. Following sepsis, patients are likely to require additional intensive care unit (ICU) visits and have higher 5-year mortality rates relative to the rest of the population [Pavon, Arnaud, et al. “Profile of the risk of death after septic shock in the present era: an epidemiologic study.” Critical care medicine 41.11 (2013): 2600-2609; Jensen, Isaac J., et al. “Sepsis-induced T cell immunoparalysis: the ins and outs of impaired T cell immunity.” The Journal of Immunology 200.5 (2018): 1543-1553].

One example of the multifactorial effects of sepsis on the body is hyperinflammation (also known as cytokine storm), which produces a deluge of pro- and anti-inflammatory mediators. Supportive therapies such as fluid therapy, antibiotics, and vasopressors, which are routinely administered once sepsis is suspected, do not remove, or treat these inflammatory mediators and therefore cannot limit the progression to immunoparalysis. As a consequence, if left untreated, this cytokine storm, also known as systemic inflammatory release syndrome (SIRS), propagates the inflammatory sequalae towards organ failure, increased susceptibility to nosocomial infections and low virulence pathogens, and possibly death. Cytokine storm and hyperinflammation can also directly cause apoptosis of immune effector cells, further weakening the immune response and contributing to immune suppression.

A sorbent technology therapy that attenuates the excessive hyperinflammatory response by removing proinflammatory cytokines, interleukins (IL), chemokines, tumor necrosis factor, interferons, lymphokines, that have been upregulated to pathological levels and present in circulating blood would limit the body's excessive compensatory anti-inflammatory response and the progression to and severity of immunoparalysis [Chousterman, Benjamin G., Filip K. Swirski, and Georg F. Weber. “Cytokine storm and sepsis disease pathogenesis.” Seminars in immunopathology. Vol. 39. No. 5. Springer Berlin Heidelberg, 2017.].

In the event that the patient recuperates from the initial sepsis episode, the immune system may not revert back to homeostasis. The immune system can remain in a state of immune suppression or immunoparalysis, for months to years, leading to significant vulnerability to low virulence pathogens and infections. In patients with immunoparalysis, there is often an excess of anti-inflammatory mediators and cytokines (e.g. IL-1 receptor antagonist, IL-4, IL-10, IL-11, IL-13), that can prevent or reduce immune system activation, function, or responsiveness. It has also been reported that immune checkpoint proteins such as CTLA-4 (Cytotoxic T-lymphocyte-associated protein 4), PD-1 (Programmed cell death protein 1 (PD-1), T-cell immunoglobulin mucin 3 (TIM-3), B- and T-lymphocyte attenuator (BTLA), natural killer cell receptor, NKG2D (natural-killer group 2, member D), lymphocyte-activation gene-3 (LAG-3), and their respective ligands, are upregulated. This can cause immune cell dysfunction, including T cell exhaustion, neutrophil and monocyte function suppression, and accelerated apoptosis. A sorbent that directly removes these inhibitory cytokines, receptors, and their ligands, as well as their soluble forms, may allow for the body to regain immune system function, thereby reversing or limiting immunoparalysis [Patera, Andriani C., et al. “Frontline Science: Defects in immune function in patients with sepsis are associated with PD-1 or PD-L1 expression and can be restored by antibodies targeting PD-1 or PD-L1.” Journal of Leukocyte Biology. 100.6 (2016): 1239-1254].

Another example of a condition that can culminate in immunoparalysis is acute-on-chronic liver failure. Studies have shown that the progression of this condition includes a dysregulated immune response and multiple organ failure. During this progression, immune cell function becomes altered—certain cell types such as dendritic cells and lymphocytes have increased apoptosis, and other cells such as neutrophils have decreased apoptosis. These cellular changes can lower or completely disable the immune system, causing immunoparalysis and immune-exhaustion [Chousterman, Benjamin G., Filip K. Swirski, and Georg F. Weber. “Cytokine storm and sepsis disease pathogenesis.” Seminars in immunopathology. Vol. 39. No. 5. Springer Berlin Heidelberg, 2017.].

Due to the immunosenescence that occurs as part of the ageing process, the risk for immunoparalysis increases. Impairments in the immune response and T-cell function is similar between immunosenescence and immunoparalysis, and both have been shown to produce increased systemic proinflammatory response as well as upregulation of immune checkpoint proteins such as PD-1 [Sokoya, T., et al. “HIV as a Cause of Immune Activation and Immunosenescence.” Mediators of inflammation (2017)].

A biomarker of immunoparalysis is the human leukocyte antigen-DR (HLA-DR). During sepsis and septic shock, monocytic HLA-DR expression is decreased [Volka, Hans-Dieter, Petra Reinkeb, and Wolf-Dietrich Dockea. “Clinical Aspects: From Systemic Inflammation to ‘Immunoparalysis’.” Cd14 in the Inflammatory Response 74 (2000): 162-177], thereby diminishing the various monocytic functions, which include neutralizing toxins and phagocytosis of pathogenic microorganisms.

### SUMMARY OF THE INVENTION

In some aspects, the invention concerns a method of treating immunoparalysis in a subject, comprising treating a subject with a therapeutically effective amount of porous biocompatible polymer sorbent comprising a range of pore diameters between about 50 Å to about 3000 Å and a pore volume between about 0.5 cc/g to about 3.0 cc/g dry polymer to the subject. The sorbent can be administered extracorporeally or administered intravenously, intramuscularly, intratumorally, intradermally, intraperitoneally, subcutaneously, orally, or nasally.

In some aspects, the invention concerns a method of treating immunoparalysis in a subject, comprising treating a subject with a therapeutically effective amount of porous biocompatible polymer sorbent comprising a range of pore diameters between about 50 Å to about 40,000 Å and a pore volume between about 0.5 cc/g to about 5.0 cc/g dry polymer to the subject. The sorbent can be administered extracorporeally or administered intravenously, intramuscularly, intratumorally, intradermally, intraperitoneally, subcutaneously, orally, or nasally.

Some sorbent are produced using at least one crosslinking agent and at least one monomer.

Preferred monomer include one or more of divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, and divinylformamide.

In some embodiments, the crosslinking agent comprises one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

Certain sorbent are produced additionally utilizing one or both of at least one dispersing agent and at least one porogen.

Some sorbents comprise a biocompatible and hemocompatible exterior coating that is covalently bound to the sorbent by free-radical grafting.

In some embodiments, the sorbent is administered prior to administration of supportive therapies. In other embodiments, the sorbent is administered at the same time as administration of supportive therapies. In yet other embodiments, the sorbent is administered following administration of supportive therapies. Supportive therapies can include antibiotics, fluid resuscitation, and vasoactive drugs.

Administration of the sorbent can improve a variety of symptoms suffered by a subject. In some embodiments, the administration of the sorbent reduces the severity of immunoparalysis. In other embodiments, the administration of the sorbent increases HLA-DR function. In yet other embodiments, the administration of the sorbent reduces the damage to the body caused by the hyperinflammatory response, thereby decreasing the patient's risk of immunoparalysis. Administration of the sorbent can also reduce T-cell exhaustion.

In some embodiments, the administration of the sorbent results in removal of one or more of a) inflammatory or anti-inflammatory mediators and b) proteins and their corresponding ligands, including soluble forms, from bodily fluid of the subject. In certain embodiments, administration of the sorbent results in partial or full restoration of immune cell function, including for example lymphocytes (e.g. T-cells, B-cells, natural killer cells), neutrophils (granulocytes), monocytes, macrophages, dendritic cells, eosinophils, and basophils. Proteins may include one or more of CTLA-4, PD-1, TIM-3, BTLA, natural killer cell receptor, NKG2D, LAG-3, and their respective ligands, including PD-L1, PD-L2, MICA, ULBP2, FasL, CTLA-4, MHC class II, and CD40L. Inflammatory or anti-inflammatory mediators may comprise one or more of a member of interleukin, cytokine, chemokine, interferon, lymphokine, tumor growth factor, complement factor, or tumor necrosis factor family. In specific embodiments, the sorbent is used to remove PD-L1.

A variety of suitable sorbents are disclosed herein. In some embodiments, the sorbent comprises one or more residues of divinylbenzene and ethylvinylbenzene, styrene, and ethylstyrene monomers.

Some aspects of the invention concerns methods of reducing sepsis and inflammation related T-cell dysfunction or other immune cell dysfunction in a subject comprising treating the subject with a therapeutically effective amount of porous biocompatible polymer sorbent comprising a range of pore diameters between about 50 Å to about 3000 Å and a pore volume between about 0.5 cc/g to about 3.0 cc/g dry polymer. The sorbent can be administered extracorporeally or administered intravenously, intramuscularly, intratumorally, intradermally, intraperitoneally, subcutaneously, orally, or nasally.

Some aspects of the invention concerns methods of reducing sepsis and inflammation related T-cell dysfunction or other immune cell dysfunction in a subject comprising treating the subject with a therapeutically effective amount of porous biocompatible polymer sorbent comprising a range of pore diameters between about 50 Å to about 40,000 Å and a pore volume between about 0.5 cc/g to about 5.0 cc/g dry polymer. The sorbent can be administered extracorporeally or administered intravenously, intramuscularly, intratumorally, intradermally, intraperitoneally, subcutaneously, orally, or nasally.

### BRIEF DESCRIPTION OF THE DRAWINGS

FIG. 1 depicts data from a benchtop evaluation showing the average percent removal of sPD-L1 using CytoSorb versus Control from bovine whole blood. The data represents mean±S.D.; each group was evaluated three times (n=3). CytoSorb 60 and 120 indicate the volume of CytoSorb used (60 mL and 120 mL, respectively). Control 60 and 120 are the controls (sham, no CytoSorb) that were evaluated at the same time as the treatment (CytoSorb) groups.

FIG. 2 depicts plots of adsorption of cytokines (time vs. % remaining) from whole blood for CytoSorb versus Control. FIG. 2A is directed to adsorption of IFN-γ. FIG. 2B is directed to adsorption of IL-6. FIG. 2C is directed to adsorption of MIP-1α. FIG. 2D is directed to adsorption of TNF-α. The percent remaining is taken from the mean±SEM of 4 runs. *p<0.05.

FIG. 3 depicts plots of adsorption of DAMPS (time vs. % remaining) from whole blood for CytoSorb versus Control. FIG. 3A is directed to adsorption of C5a. FIG. 3B is directed to adsorption of HMGB-1. FIG. 3C is directed to adsorption of procalcitonin. FIG. 3D is directed to adsorption of S100-A8. The percent remaining is taken from the mean±SEM of 4 runs. *p<0.05.

FIG. 4 depicts plots of adsorption of bacterial PAMPS (time vs. % remaining) from whole blood (spiked with S. pyogenic exotoxin B, Staph TSST-1 or serum with Staph aureus alpha-toxin) for CytoSorb versus Control. FIG. 4A is directed to adsorption of α-toxin. FIG. 4B is directed to adsorption of SpeB. FIG. 4C is directed to adsorption of TSST-1. The percent remaining is taken from the mean±SEM of 4 runs. *p<0.05.

### DETAILED DESCRIPTION OF ILLUSTRATIVE EMBODIMENTS

The invention concerns immunomodulating therapies for the treatment of conditions or critical injuries that are affected by a decrease in immune system function, immunosuppression, anergy, or immunoparalysis. A decrease in immune system function can lead to compromised healing and recovery, poor response to infectious stimuli and an increased risk of infection and sepsis, poor immune response to vaccinations, an increased risk of malignancy, abnormal tissue remodeling, and other complications. This can contribute to the poor short and long-term outcomes seen in critical illnesses, including but not limited to conditions such as infection, sepsis and septic-shock, trauma, burn injury, liver disease, pancreatitis, complications of surgery, atherosclerosis, allograft vascular disease, encephalomyelitis, stroke, sepsis and viral myocarditis, cardiac ischemia-reperfusion (IR) injury myocardial infarction, acute-on-chronic liver failure, as well as treatment for age related conditions such as immunosenescence [Davies, Roger, Kieran O'Dea, and Anthony Gordon. “Immune therapy in sepsis: Are we ready to try again?.” Journal of the Intensive Care Society 19.4 (2018): 326-344; Baban, Babak, et al. “Upregulation of programmed death-1 and its ligand in cardiac injury models: interaction with GADD153.” PloS one 10.4 (2015): e0124059]. Application of sorbent technologies to modulate the immune response during critical injuries and conditions (including sepsis), is a way to minimize the severity or effects of immunosuppression during and/or following these critical conditions, as a way to minimize the severity of immunoparalysis during and/or following these critical conditions, and as a way to allow the immune response to revert back to homeostasis during and following these critical conditions.

As required, detailed embodiments of the present invention are disclosed herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the invention to be better understood. However, they are given merely by way of guidance and do not imply any limitation.

The present invention may be understood more readily by reference to the following detailed description taken in connection with the accompanying figures and examples, which form a part of this disclosure. It is to be understood that this invention is not limited to the specific materials, devices, methods, applications, conditions or parameters described and/or shown herein, and that the terminology used herein is for the purpose of describing particular embodiments by way of example only and is not intended to be limiting of the claimed invention. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention, which are, for clarity, described herein in the context of separate embodiments, may also be provided in combination in a single embodiment. Conversely, various features of the invention that are, for brevity, described in the context of a single embodiment, may also be provided separately or in any subcombination. Further reference to values stated in ranges includes each and every value and combination of values within that range.

The immune system is tightly regulated by pro- and anti-inflammatory mediators, cells, and other factors to prevent either hyperinflammation or immune suppression. In critical illness, however, immune system homeostasis is often compromised with periods of hyperinflammation and severe immunosuppression, anergy, or immunoparalysis. Hyperinflammation can paradoxically cause immune suppression later in the disease by, for example, triggering apoptosis of immune effector cells, causing upregulation of endothelial cell adhesion molecules that abnormally sequesters infection-fighting leukocytes to the endothelium of blood vessels in healthy organs and away from the site of infection, and a rebound anti-inflammatory response, also called the compensatory anti-inflammatory response syndrome (CARS). CARS is often characterized by severe immune suppression due to, for example, the loss of immune effector cells, and upregulation of immune suppressor or regulatory cells (e.g. T regulatory cells), and the overabundance of anti-inflammatory mediators. For example, one class of anti-inflammatory mediators include anti-inflammatory cytokines (e.g. interleukins, interferons, others). Another class are immune checkpoint proteins. Immune checkpoints regulate the immune system's response to infections, malignancies, and prevent autoimmunity. For example, the immune checkpoint protein PD-1 is expressed on activated T-cells and plays a central role in down regulating these cells. Activation of PD-1 by its membrane-bound ligands, PD-L1 and PD-L2, and soluble counterparts, sPD-L1 and sPD-L2, lead to apoptosis of the T-cell, leading to immune suppression. The PD-1/PD-L1 pathway is one of the most widely studied, and the upregulation of these proteins has been associated with immunoparalysis. PD-1 is expressed in a number of cells, including T cells, B cells, and NK cells. PD-L1 expression has been confirmed in monocytes, neutrophils, and others. In patients with sepsis induced immunosuppression, a number of checkpoint inhibitor proteins are upregulated, thereby altering immune cell function.

Hyperinflammatory responses, also known as cytokine storm, are episodes of highly elevated pro- and anti-inflammatory systemic immune responses due to a variety of conditions, including trauma, disease, auto-immune syndromes, and medical interventions.

### Definitions

The following definitions are intended to assist in understanding the present invention.

Some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and claims of this specification, the words “comprise” and “contain” and variations of the words, for example “comprising” and “comprises”, mean “including but not limited to”, and are not intended to (and do not) exclude other components. As used herein and in the appended claims, the singular forms “a,” “an,” and “the” include plural reference unless the context clearly dictates otherwise.

The term “subject” means any animal, particularly a mammal, most particularly a human, who will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more particularly a human.

The term “immunoparalysis” describes a phase in which immune homeostasis is not restored, leading to immunosuppression. It can also be referred to as anergy.

The term “biocompatible” is defined to mean the sorbent is capable of coming in contact with physiologic fluids, living tissues, or organisms, without producing unacceptable clinical changes during the time that the sorbent is in contact with the physiologic fluids, living tissues, or organisms.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physical parameter including those which may not be discernible by the patient. In a further embodiment, “treat”, “treating”, or “treatment” refers to modulating the disease, condition, syndrome, or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome, or disorder.

The term “hemocompatible” is defined as a condition whereby a biocompatible material when placed in contact with whole blood or blood plasma results in clinically acceptable physiologic changes.

As used herein, the term “physiologic fluids” are liquids that originate from the body and can include, but are not limited to, nasopharyngeal, oral, esophageal, gastric, pancreatic, hepatic, pleural, pericardial, peritoneal, intestinal, prostatic, seminal, vaginal secretions, as well as tears, saliva, lung, or bronchial secretions, mucus, bile, blood, lymph, plasma, serum, synovial fluid, cerebrospinal fluid, urine, and interstitial, intracellular, and extracellular fluid, such as fluid that exudes from burns or wounds.

As used herein, the term “sorbent” includes adsorbents and absorbents.

For purposes of this invention, the term “sorb” is defined as “taking up and binding by absorption and/or adsorption”.

The term “supportive therapies” means conventional treatments used in combating symptoms of immunoparalysis. Examples of supportive therapies include antibiotics, vasopressors, mechanical ventilation and extracorporeal membrane oxygenation (in cases of acute respiratory syndrome secondary to sepsis), hemodialysis and hemofiltration (due to kidney injury), and fluid therapy.

For the purposes of this invention, the term “perfusion” is defined as passing a physiologic fluid, once through or by way of a suitable extracorporeal circuit, through a device containing the porous polymeric adsorbent to remove toxic molecules from the fluid.

The term “hemoperfusion” is a special case of perfusion where the physiologic fluid is blood.

The term “dispersant” or “dispersing agent” is defined as a substance that imparts a stabilizing effect upon a finely divided array of immiscible liquid droplets suspended in a fluidizing medium.

The term “heparin mimicking polymer” refers to any polymer that possesses the same anticoagulant and/or antithrombogenic properties as heparin. In some embodiments, the sorbent can act as a heparin mimicking polymer by having functional groups selected from —SO₃H, —COOH and —OH (or salts thereof) on the sorbent surface. These functional groups may be attached to the sorbent polymer by means known to those skilled in the art. See, for example, Ran, et al. Macromol. Biosci. 2012, 12(1), 116-25. Suitable salts include sodium and potassium salts such as —SO₃⁻M⁺, —COO⁻M⁺, and —O⁻M⁺ where M is Na or K.

The term “macroreticular synthesis” is defined as a polymerization of monomers into polymer in the presence of an inert precipitant which forces the growing polymer molecules out of the monomer liquid at a certain molecular size dictated by the phase equilibria to give solid nanosized microgel particles of spherical or almost spherical symmetry packed together to give a bead with physical pores of an open cell structure [U.S. Pat. No. 4,297,220, Meitzner and Oline, Oct. 27, 1981; R. L. Albright, Reactive Polymers, 4, 155-174(1986)].

The term “hypercrosslinked” describes a polymer in which the single repeating unit has a connectivity of more than two. Hypercrosslinked polymers are prepared by crosslinking swollen, or dissolved, polymer chains with a large number of rigid bridging spacers, rather than copolymerization of monomers. Crosslinking agents may include bis(chloromethyl) derivatives of aromatic hydrocarbons, methylol, monochlorodimethyl ether, and other bifunctional compounds that react with the polymer in the presence of Friedel-Crafts catalysts [Tsyurupa, M. P., Z. K. Blinnikova, N. A. Proskurina, A. V. Pastukhov, L. A. Pavlova, and V. A. Davankov. “Hypercrosslinked Polystyrene: The First Nanoporous Polymeric Material.” Nanotechnologies in Russia 4 (2009): 665-75.]

The term “pathogen-associated molecular pattern molecules” (PAMPS) are molecules derived from microorganisms that are recognized by cells of the innate immune system. These molecules have small molecular motifs conserved within a class of microbes that are recognized by toll-like receptor and pattern recognition receptors that initiate and perpetuate a pathogen induced inflammatory response.

The term “damage-associated molecular pattern molecules” (DAMPS) are host biomolecules released by stressed cells that initiate and perpetuate inflammation in response to trauma, ischemia, and tissue damage either in the absence or presence of pathogenic infection.

### Sorbents

One embodiment of the invention is a porous polymer sorbent. The porous polymer sorbent is configured to sorb cytokines, interleukins, chemokines, tumor necrosis factor, interferons, lymphokines, soluble checkpoint inhibitor ligands, and other proteins involved in immunoparalysis from a physiologic fluid from a patient. In certain embodiments, the sorbent is configured to remove such proteins from the blood of a patient.

Some preferred polymers comprise residues from one or more monomers, or containing monomers, or mixtures thereof, selected from acrylonitrile, allyl glycidyl ether, butyl acrylate, butyl methacrylate, cetyl acrylate, cetyl methacrylate, 3,4-dihydroxy-1-butene, dipentaerythritol diacrylate, dipentaerythritol dimethacrylate, dipentaerythritol tetraacrylate, dipentaerythritol tetramethacrylate, dipentaerythritol triacrylate, dipentaerythritol trimethacrylate, divinylbenzene, divinylformamide, divinylnaphthalene, divinylsulfone, 3,4-epoxy-1-butene, 1,2-epoxy-9-decene, 1,2-epoxy-5-hexene, ethyl acrylate, ethyl methacrylate, ethylstyrene, ethylvinylbenzene, glycidyl methacrylate, methyl acrylate, methyl methacrylate, octyl acrylate, octyl methacrylate, pentaerythritol diacrylate, pentaerythritol dimethacrylate, pentaerythritol tetraacrylate, pentaerythritol tetramethacrylate, pentaerythritol triacrylate, pentaerythritol trimethacrylate, styrene, trimethylolpropane diacrylate, trimethylolpropane dimethacrylate, trimethylolpropane triacrylate, trimethylolpropane trimethacrylate, trivinylbenzene, trivinylcyclohexane, vinyl acetate, vinylbenzyl alcohol, 4-vinyl-1-cyclohexene 1,2-epoxide, vinylformamide, vinylnaphthalene, 2-vinyloxirane, and vinyltoluene.

Some embodiments of the invention use an organic solvent and/or polymeric porogen as the porogen or pore-former, and the resulting phase separation induced during polymerization yield porous polymers. Some preferred porogens are selected from, or mixtures comprised of any combination of, benzyl alcohol, cyclohexane, cyclohexanol, cyclohexanone, decane, dibutyl phthalate, di-2-ethylhexyl phthalate, di-2-ethylhexylphosphoric acid, ethylacetate, 2-ethyl-1-hexanoic acid, 2-ethyl-1-hexanol, n-heptane, n-hexane, isoamyl acetate, isoamyl alcohol, n-octane, pentanol, poly(propylene glycol), polystyrene, poly(styrene-co-methyl methacrylate), tetraline, toluene, tri-n-butylphosphate, 1,2,3-trichloropropane, 2,2,4-trimethylpentane, and xylene.

In yet another embodiment, the dispersing agent is selected from a group consisting of hydroxyethyl cellulose, hydroxypropyl cellulose, poly(diethylaminoethyl acrylate), poly(diethylaminoethyl methacrylate), poly(dimethylaminoethyl acrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(vinyl alcohol), salts of poly(acrylic acid), salts of poly(methacrylic acid) and mixtures thereof.

Preferred sorbents are biocompatible. In another further embodiment, the polymer is biocompatible. In yet another embodiment, the polymer is hemocompatible. In still a further embodiment, the biocompatible polymer is hemocompatible. In still a further embodiment, the geometry of the polymer is a spherical bead.

In another embodiment, the biocompatible polymer comprises poly(N-vinylpyrrolidone).

In another embodiment, the biocompatible polymer comprises 1,2-diols. In another embodiment, the biocompatible polymer comprises 1,3-diols

In another further embodiment, the biocompatible polymer comprises heparin mimicking polymers.

The coating/dispersant on the poly(styrene-co-divinylbenzene) resin will imbue the material with improved biocompatibility.

In still yet another embodiment, a group of cross-linkers consisting of dipentaerythritol diacrylates, dipentaerythritol dimethacrylates, dipentaerythritol tetraacrylates, dipentaerythritol tetramethacrylates, dipentaerythritol triacrylates, dipentaerythritol trimethacrylates, divinylbenzene, divinylformamide, divinylnaphthalene, divinylsulfone, pentaerythritol diacrylates, pentaerythritol dimethacrylates, pentaerythritol tetraacrylates, pentaerythritol tetramethacrylates, pentaerythritol triacrylates, pentaerythritol trimethacrylates, trimethylolpropane diacrylate, trimethylolpropane dimethacrylate, trimethylolpropane triacrylate, trimethylolpropane trimethacrylate, trivinylbenzene, trivinylcyclohexane and mixtures thereof can be used in formation of a hemocompatible hydrogel coating.

In some embodiments, the polymer is a polymer comprising at least one crosslinking agent and at least one dispersing agent. The dispersing agent may be biocompatible. The dispersing agents can be selected from chemicals, compounds or materials such as hydroxyethyl cellulose, hydroxypropyl cellulose, poly(diethylaminoethyl acrylate), poly(diethylaminoethyl methacrylate), poly(dimethylaminoethyl acrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(vinyl alcohol), salts of poly(acrylic acid), salts of poly(methacrylic acid) and mixtures thereof; the crosslinking agent selected from a group consisting of dipentaerythritol diacrylates, dipentaerythritol dimethacrylates, dipentaerythritol tetraacrylates, dipentaerythritol tetramethacrylates, dipentaerythritol triacrylates, dipentaerythritol trimethacrylates, divinylbenzene, divinylformamide, divinylnaphthalene, divinylsulfone, pentaerythritol diacrylates, pentaerythritol dimethacrylates, pentaerythritol tetraacrylates, pentaerythritol tetramethacrylates, pentaerythritol triacrylates, pentaerythritol trimethacrylates, trimethylolpropane diacrylate, trimethylolpropane dimethacrylate, trimethylolpropane triacrylate, trimethylolpropane trimethacrylate, trivinylbenzene, trivinylcyclohexane and mixtures thereof. Preferably, the polymer is developed simultaneously with the formation of the coating, wherein the dispersing agent is chemically bound or entangled on the surface of the polymer.

In still another embodiment, the biocompatible polymer coating is selected from a group consisting of poly(diethylaminoethyl methacrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(N-vinylpyrrolidone), poly(vinyl alcohol), salts of poly(acrylic acid), salts of poly(methacrylic acid) and mixtures thereof.

In still another embodiment, the biocompatible oligomer coating is selected from a group consisting of poly(diethylaminoethyl methacrylate), poly(dimethylaminoethyl methacrylate), poly(hydroxyethyl acrylate), poly(hydroxyethyl methacrylate), poly-(hydroxypropyl acrylate), poly(hydroxypropyl methacrylate), poly(N-vinylpyrrolidone), poly(vinyl alcohol), salts of poly(acrylic acid), salts of poly(methacrylic acid) and mixtures thereof.

Some present biocompatible sorbent compositions are comprised of a plurality of pores. The biocompatible sorbents are designed to adsorb a broad range of toxins from less than 0.5 kDa to 1,000 kDa. While not intending to be bound by theory, it is believed the sorbent acts by sequestering molecules of a predetermined molecular weight within the pores. The size of a molecule that can be sorbed by the polymer will increase as the pore size of the polymer increases. Conversely, as the pore size is increased beyond the optimum pore size for adsorption of a given molecule, adsorption of said protein may or will decrease.

In certain embodiments, the structure of some polymers is such that the total pore volume of pore size in the range of 50 Å to 3000 Å is between 0.5 cc/g to 3.0 cc/g dry polymer.

In some embodiments, the polymer has a pore structure such that the total pore volume of pore size in the range of 50 Å to 3000 Å is greater than 0.5 cc/g to 3.0 cc/g dry polymer; wherein the ratio of pore volume between 50 Å to 3,000 Å (pore diameter) to pore volume between 500 Å to 3,000 Å (pore diameter) of the polymer is smaller than 200:1; and the ratio of pore volume between 50 Å to 3,000 Å (pore diameter) to pore volume between 1,000 Å to 3,000 Å (pore diameter) of the polymer is greater than 20:1.

In certain embodiments, the solid form is porous. Some solid forms are characterized as having a pore structure having a total volume of pore sizes in the range of from 10 Å to 40,000 Å greater than 0.1 cc/g and less than 5.0 cc/g dry polymer.

In certain embodiments, the sorbent comprises a pore structure such that the total pore volume of pore size in the range of 50 Å to 40,000 Å is greater than 0.5 cc/g to 5.0 cc/g dry polymer; wherein the ratio of pore volume between 50 Å to 40,000 Å (pore diameter) to pore volume between 1,000 Å to 10,000 Å (pore diameter) of the sorbent is smaller than 2:1.

Sorbents according to the instant disclosure that may be suitable for use in immunotherapies are biocompatible polymer sorbents having the following characteristics: a range of pore diameters between about 50 Å to about 3000 Å, alternatively from about 60 Å to about 2000 Å, alternatively from about 75 Å to about 3000 Å, alternatively from about 50 Å to about 2000 Å, alternatively from about 100 Å to about 1500 Å; and a pore volume between about 0.5 cc/g to about 3.0 cc/g dry polymer.

In certain embodiments, the structure of some polymers is such that the total pore volume of pore size in the range of 50 Å to 3000 Å is between 0.5 cc/g to 3.0 cc/g dry polymer. In some embodiments, the polymer has a pore structure such that the total pore volume of pore size in the range of 50 Å to 3000 Å is greater than 0.5 cc/g to 3.0 cc/g dry polymer; wherein the ratio of pore volume between 50 Å to 3,000 Å (pore diameter) to pore volume between 500 Å to 3,000 Å (pore diameter) of the polymer is smaller than 200:1; and the ratio of pore volume between 50 Å to 3,000 Å (pore diameter) to pore volume between 1,000 Å to 3,000 Å (pore diameter) of the polymer is greater than 20:1.

In certain embodiments, the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

In certain embodiments, the sorbent has a pore structure having a total volume of pore sizes in the range of from 10 Å to 10,000 Å is greater than 0.5 cc/g to 3.0 cc/g dry polymer; wherein the ratio of pore volume between 10 Å to 3,000 Å in diameter to pore volume between 500 Å to 3,000 Å in diameter of the said cross-linked polymeric material is smaller than 7:1 and wherein the ratio of pore volume between 10 Å to 3,000 Å in diameter to pore volume between 10 Å to 6,000 Å in diameter of said cross-linked polymeric material is less than 2:1.

In some embodiments, the sorbent has:
a) a pore structure wherein at least ⅓ of the pore volume in pores having diameters between 50 Å and 40,000 Å is in pores having diameters between 100 Å and 1,000 Å; or
(b) a pore structure wherein at least ½ of the pore volume in pores having diameters between 50 Å and 40,000 Å is in pores having diameters between 1000 Å and 10,000 Å; or
(c) a pore structure wherein at least ⅓ of the pore volume in pores having diameters between 50 Å and 40,000 Å is in pores having diameters between 10,000 Å and 40,000 Å.

In certain other methods, the solid form is nonporous.

In certain embodiments, the polymers can be made in bead form having a diameter in the range of 0.1 micrometers to 2 centimeters. Certain polymers are in the form of powder, beads or other regular or irregularly shaped particulates.

In some embodiments, the plurality of solid forms comprises particles having a diameter in the range for 0.1 micrometers to 2 centimeters.

In some embodiments, sorbents include cross-linked polymeric material derived from the reaction of a cross-linker with one or more of the following polymerizable monomers, then subsequently epoxidized and ring-opened to form a polyol: acrylonitrile, allyl glycidyl ether, butyl acrylate, butyl methacrylate, cetyl acrylate, cetyl methacrylate, 3,4-dihydroxy-1-butene, dipentaerythritol diacrylate, dipentaerythritol dimethacrylate, dipentaerythritol tetraacrylate, dipentaerythritol tetramethacrylate, dipentaerythritol triacrylate, dipentaerythritol trimethacrylate, divinylbenzene, divinylformamide, divinylnaphthalene, divinylsulfone, 3,4-epoxy-1-butene, 1,2-epoxy-9-decene, 1,2-epoxy-5-hexene, ethyl acrylate, ethyl methacrylate, ethylstyrene, ethylvinylbenzene, glycidyl methacrylate, methyl acrylate, methyl methacrylate, octyl acrylate, octyl methacrylate, pentaerythritol diacrylate, pentaerythritol dimethacrylate, pentaerythritol tetraacrylate, pentaerythritol tetramethacrylate, pentaerythritol triacrylate, pentaerythritol trimethacrylate, styrene, trimethylolpropane diacrylate, trimethylolpropane dimethacrylate, trimethylolpropane triacrylate, trimethylolpropane trimethacrylate, trivinylbenzene, trivinylcyclohexane, vinyl acetate, vinylbenzyl alcohol, 4-vinyl-1-cyclohexene 1,2-epoxide, vinylformamide, vinylnaphthalene, 2-vinyloxirane, and vinyltoluene. In preferred sorbents, the formed polyol is a diol.

In another embodiment, polymeric sorbents are prepared from the reaction of a cross-linker with vinyl acetate and subsequently modified to form a bead containing polyol groups. The reaction may be a copolymerization, or a one-pot reaction in which vinyl acetate is added once initial polymerization has nearly completed, utilizing unused initiator to begin a second free-radical polymerization to add vinyl acetate groups to the surface of the polymer beads. The subsequent modification of the vinyl acetate containing polymer includes, in order: hydrolysis to convert acetate groups into hydroxyl groups, reaction with epichlorohydrin to form polymer beads containing epoxide groups, and ring-opening to convert epoxide groups into polyol groups. In preferred embodiments, polyols are diols.

Some embodiments of the invention involve direct synthesis of polymeric beads containing epoxide groups, followed by ring-opening of epoxide groups to form polyols. One or more of the following polymerizable vinyl monomer containing epoxide groups can be polymerized in the presence of cross-linker and monomer to yield polymeric beads containing above mentioned functionalities: allyl glycidyl ether, 3,4-dihydroxy-1-butene, 3,4-epoxy-1-butene, 1,2-epoxy-9-decene, 1,2-epoxy-5-hexene, glycidyl methacrylate, 4-vinyl-1-cyclohexene 1,2-epoxide, and 2-vinyloxirane. Vinyl monomers containing epoxide groups can also be copolymerized with hemocompatible monomer (NVP. 2-HEMA, etc.) to yield hemocompatible beads containing epoxide groups. In preferred embodiments, the polyols are diols.

Still other embodiments consist of hypercrosslinked polymeric sorbents containing polyol groups on the beads' surfaces. This can be accomplished via free-radical or SN2 type chemistries. The chemical modification of the surface of sorbent beads, which is the case in the above modification, is facilitated by the remarkable peculiarity of the hypercrosslinked polystyrene; namely, that the reactive functional groups of the polymer are predominantly located on its surface. The hypercrosslinked polystyrene is generally prepared by crosslinking polystyrene chains with large amounts of bifunctional compounds, in particular, those bearing two reactive chloromethyl groups. The latter alkylate, in a two-step reaction, two phenyl groups of neighboring polystyrene chains according to Friedel-Crafts reaction, with evolution of two molecules of HCl and formation of a cross bridge. During the crosslinking reaction, the three-dimensional network formed acquires rigidity. This property gradually reduces the rate of the second step of the crosslinking reaction, since the reduced mobility of the second pendant functional group of the initial crosslinking reagent makes it more and more difficult to add an appropriate second partner for the alkylation reaction. This is especially characteristic of the second functional groups that happen to be exposed to the surface of the bead. Therefore, of the pendant unreacted chloromethyl groups in the final hypercrosslinked polymer, the largest portion, if not the majority of the groups, are located on the surface of the bead (or on the surface of pores). This circumstance makes it possible to predominantly modify the surface of the polymer beads by involving the above chloromethyl groups into various chemical reactions that allow attachment of biocompatible and hemocompatible monomers, and/or cross-linkers or low molecular weight oligomers. The subsequent introduction of hydroxyl groups, followed by reaction with epichlorohydrin, results in the polymer sorbent containing epoxide groups on the beads' surfaces. These epoxide groups can then be ring-opened to form polyol groups. In some preferred embodiments, the polyols are diols.

In other embodiments, hypercrosslinked polystyrene containing pendant unreacted chloromethyl groups is directly modified in the presence of one or more of the following reagents to form sorbent polymer beads containing polyols on the beads' surfaces (or on the surface of pores): (±)-3-amino-1,2-propanediol, glycerol, and other polyols. In preferred embodiments, the polyols are diols.

Still in other embodiments, the surface coating biocompatibility and hemocompatibility agent, poly(vinyl alcohol), also acts as the polyol functional group.

In some other embodiments, sorbents include cross-linked polymeric material derived from the reaction of a cross-linker with one or more of the following polymerizable monomers, then subsequently reacted with a polymerizable zwitterionic monomer in the presence of a free radical initiator: acrylonitrile, allyl glycidyl ether, butyl acrylate, butyl methacrylate, cetyl acrylate, cetyl methacrylate, 3,4-dihydroxy-1-butene, dipentaerythritol diacrylate, dipentaerythritol dimethacrylate, dipentaerythritol tetraacrylate, dipentaerythritol tetramethacrylate, dipentaerythritol triacrylate, dipentaerythritol trimethacrylate, divinylbenzene, divinylformamide, divinylnaphthalene, divinylsulfone, 3,4-epoxy-1-butene, 1,2-epoxy-9-decene, 1,2-epoxy-5-hexene, ethyl acrylate, ethyl methacrylate, ethylstyrene, ethylvinylbenzene, glycidyl methacrylate, methyl acrylate, methyl methacrylate, octyl acrylate, octyl methacrylate, pentaerythritol diacrylate, pentaerythritol dimethacrylate, pentaerythritol tetraacrylate, pentaerythritol tetramethacrylate, pentaerythritol triacrylate, pentaerythritol trimethacrylate, styrene, trimethylolpropane diacrylate, trimethylolpropane dimethacrylate, trimethylolpropane triacrylate, trimethylolpropane trimethacrylate, trivinylbenzene, trivinylcyclohexane, vinyl acetate, vinylbenzyl alcohol, 4-vinyl-1-cyclohexene 1,2-epoxide, vinylformamide, vinylnaphthalene, 2-vinyloxirane, and vinyltoluene. Polymerizable zwitterionic monomers include one, or more, of the following: 2-Acrylamido-2-methyl-1-propanesulfonic acid sodium salt, [3-(Acryloylamino)propyl]-trimethylammonium chloride, 3-[[2-(Acryloyloxy)ethyl]-dimethylammonio]-propionate, [2-(Acryloyloxy)ethyl]-dimethyl-(3-sulfopropyl)-ammonium hydroxide, 2-Acryloyloxyethyl phosphorylcholine, [3-(Methacryloylamino)propyl]-trimethylammonium chloride, 3-[[2-(Methacryloyloxy)ethyl]-dimethylammonio]-propionate, [2-(Methacryloyloxy)ethyl]-dimethyl-(3-sulfopropyl)-ammonium hydroxide, and 2-Methacryloyl-oxyethyl phosphorylcholine.

In one embodiment, the polymers of this invention are made by suspension polymerization in a formulated aqueous phase with free radical initiation in the presence of aqueous phase dispersants that are selected to provide a biocompatible and a hemocompatible exterior surface to the formed polymer beads. In some embodiments, the beads are made porous by the macroreticular synthesis with an appropriately selected porogen (pore forming agent) and an appropriate time-temperature profile for the polymerization in order to develop the proper pore structure.

In another embodiment, polymers made by suspension polymerization can be made biocompatible and hemocompatible by further grafting of biocompatible and hemocompatible monomers or low molecular weight oligomers. It has been shown that the radical polymerization procedure does not consume all the vinyl groups of DVB introduced into copolymerization. On average, about 30% of DVB species fail to serve as crosslinking bridges and remain involved in the network by only one of two vinyl groups. The presence of a high amount of pendant vinyl groups is therefore a characteristic feature of the adsorbents. It can be expected that these pendant vinyl groups are preferably exposed to the surface of the polymer beads and their macropores, if present, should be readily available to chemical modification. The chemical modification of the surface of DVB-copolymers relies on chemical reactions of the surface-exposed pendant vinyl groups and aims at converting these groups into more hydrophilic functional groups. This conversion via free radical grafting of monomers and/or cross-linkers or low molecular weight oligomers provides the initial hydrophobic adsorbing material with the property of hemocompatibility.

In yet another embodiment, the radical polymerization initiator is initially added to the dispersed organic phase, not the aqueous dispersion medium as is typical in suspension polymerization. During polymerization, many growing polymer chains with their chain-end radicals show up at the phase interface and can initiate the polymerization in the dispersion medium. Moreover, the radical initiator, like benzoyl peroxide, generates radicals relatively slowly. This initiator is only partially consumed during the formation of beads even after several hours of polymerization. This initiator easily moves toward the surface of the bead and activates the surface exposed pendant vinyl groups of the divinylbenzene moiety of the bead, thus initiating the graft polymerization of other monomers added after the reaction has proceeded for a period of time. Therefore, free-radical grafting can occur during the transformation of the monomer droplets into polymer beads thereby incorporating monomers and/or cross-linkers or low molecular weight oligomers that impart biocompatibility or hemocompatibility as a surface coating.

In some embodiments, impurities are removed using hemoperfusion and perfusion devices. The hemoperfusion and perfusion devices consist of a packed bead bed of the polymer beads in a flow-through container fitted with either a retainer screen at both the exit end and the entrance end to maintain the bead bed inside the container, or with a subsequent retainer screen to collect the beads after mixing. The hemoperfusion and perfusion operations are performed by passing the whole blood, blood plasma or physiologic fluid through the packed bead bed. During the perfusion through the bead bed, the toxic molecules are retained by sorption, torturous path, and/or pore capture, while the remainder of the fluid and intact cell components pass through essentially unchanged in concentration.

In some other embodiments, an in-line filter is comprised of a packed bead bed of the polymer beads in a flow-through container, fitted with a retainer screen at both the exit end and the entrance end to maintain the bead bed inside the container. Biological/physiologic fluids are passed from a storage bag once-through the packed bead bed via gravity, during which the toxic molecules are retained by sorption, torturous path, and/or pore capture, while the remainder of the fluid and intact cell components pass through essentially unchanged in concentration.

In certain other embodiments, the sorbent is administered as a pharmaceutical composition. The pharmaceutical compositions may also be administered in tablets, capsules, gel capsules, slurries, suspensions, and the like. Manufacture of such pharmaceutical compositions is well known in the art.

Penetration enhancers may also be used in the instant pharmaceutical compositions. Such enhancers include surfactants, fatty acids, bile salts, chelating agents, and non-chelating non-surfactants and are generally known in the art.

In some preferred embodiments, the administration of sorbent polymer is oral, or rectal, or via a feeding tube or any combination therein.

The polymers of the present invention can be administered once to a patient or in multiple doses.

Polymers useful in the invention may be supplied as a slurry, suspension or reconstituted from the dry state into a slurry or suspension. In some embodiments, the polymer may be supplied as a slurry or suspension packaged in either single dose or multidose bottles for oral administration. In other embodiments, the polymer may be supplied as a slurry or suspension packaged in either single dose or multidose bottles for administration by enema or feeding tube or any combination therein. In certain embodiments, the polymer is supplied as a dry powder capable of being wetted externally or in the alimentary canal with or without the addition of wetting agents such as ethyl alcohol.

The polymer may be supplied as tablet, capsule or suppository packaged in bottles or blister packs for administration. Depending on the use, the polymer may be sterile or non-sterile. The polymer may be sterilized by standard methods. Such methods are well known to those skilled in the art.

Certain polymers useful in the invention (as is or after further modification) are macroporous polymers prepared from the polymerizable monomers of styrene, divinylbenzene, ethylvinylbenzene, and the acrylate and methacrylate monomers such as those listed below by manufacturer. Rohm and Haas Company, (now part of Dow Chemical Company): macroporous polymeric sorbents such as Amberlite™ XAD-1, Amberlite™ XAD-2, Amberlite™ XAD-4, Amberlite™ XAD-7, Amberlite™ XAD-7HP, Amberlite™ XAD-8, Amberlite™ XAD-16, Amberlite™ XAD-16 HP, Amberlite™ XAD-18, Amberlite™ XAD-200, Amberlite™ XAD-1180, Amberlite™ XAD-2000, Amberlite™ XAD-2005, Amberlite™ XAD-2010, Amberlite™ XAD-761, and Amberlite™ XE-305, and chromatographic grade sorbents such as Amberchrom™ CG 71,s,m,c, Amberchrom™ CG 161,s,m,c, Amberchrom™ CG 300,s,m,c, and Amberchrom™ CG 1000,s,m,c. Dow Chemical Company: Dowex™ Optipore™ L-493, Dowex™ Optipore™ V-493, Dowex™ Optipore™ V-502, Dowex™ Optipore™ L-285, Dowex™ Optipore™ L-323, and Dowex™ Optipore™ V-503. Lanxess (formerly Bayer and Sybron): Lewatit™ VPOC 1064 MD PH, Lewatit™ VPOC 1163, Lewatit™ OC EP 63, Lewatit™ S 6328A, Lewatit™ OC 1066, and Lewatit™60/150 MIBK. Mitsubishi Chemical Corporation: Diaion™ HP 10, Diaion™ HP 20, Diaion™ HP 21, Diaion™ HP 30, Diaion™ HP 40, Diaion™ HP 50, Diaion™ SP70, Diaion™ SP 205, Diaion™ SP 206, Diaion™ SP 207, Diaion™ SP 700, Diaion™ SP 800, Diaion™ SP 825, Diaion™ SP 850, Diaion™ SP 875, Diaion™ HP 1MG, Diaion™ HP 2MG, Diaion™ CHP 55A, Diaion™ CHP 55Y, Diaion™ CHP 20A, Diaion™ CHP 20Y, Diaion™ CHP 2MGY, Diaion™ CHP 20P, Diaion™ HP 20SS, Diaion™ SP 20SS, Diaion™ SP 207SS. Purolite Company: Purosorb™ AP 250 and Purosorb™ AP 400, and Kaneka Corp. Lixelle beads.

Other certain polymers useful in the invention (as is or after further modification) are cellulosic porous materials. Such modifications could include the addition of lipophilic substrates that comprise aryl or alkyl groups, along with polyol or zwitterionic substrates, added via free-radical or SN2 type chemistries.

### Methods of Treatment Use of Sorbent in Immunomodulatory Therapies

Embodiments of the invention are directed to immunomodulatory therapies to treat conditions or critical injuries affected by the upregulation of immune checkpoint proteins, leading to immunoparalysis. Examples of such conditions include, but are not limited to, sepsis and septic-shock, trauma, burn injury, liver disease, pancreatitis, atherosclerosis, allograft vascular disease, encephalomyelitis, stroke, sepsis and viral myocarditis, cardiac ischemia-reperfusion (IR) injury myocardial infarction, acute-on-chronic liver failure, as well as treatment for age related conditions such as immunosenescence. The immunomodulatory therapies utilize the sorbents of the instant disclosure alone or in combination with supportive therapies, such as e.g. checkpoint inhibitors, monoclonal antibodies, and/or bi-specific T-cell engagers. When the sorbents are used in combination with supportive therapies, the supportive therapies may be administered before, concurrently, or after the sorbent. When used in the methods of the intention, it is contemplated that the sorbent removes one or more immune checkpoint protein. In one embodiment, it is contemplated that the sorbent removes PD-L1 and/or PD-L2, and/or their soluble counterparts, sPD-L1 and/or PD-L2.

When used in immunomodulatory therapies, the sorbent may reduce the severity of immunoparalysis and/or increases HLA-DR function. Alternatively, use of the sorbent reduces the damage to the body caused by the hyperinflammatory response, thereby decreasing the patient's risk of immunoparalysis. In another embodiment, the sorbent may be used to reduce T-cell exhaustion.

In alternate embodiments, the sorbent may be used to remove one or more of a) cytokine and b) soluble ligand from bodily/physiologic fluid of the subject. Exemplary soluble ligands include one or more of PD-L1, PD-L2, sMICA, SULBP2, sFasL, sCTLA-4, MHC class II, and sCD40L. Exemplary cytokines include one or more of a member of interleukin, chemokine, interferon, lymphokine, tumor growth factor, or tumor necrosis factor family. In one embodiment, the soluble ligand is PD-L1 and/or PD-L2.

When used in immunosorbent therapy, use of the sorbent may result in a decrease of inflammatory mediators in the subject. In one embodiment, use of the sorbent results in removal of inflammatory mediators. In another embodiment, the sorbent is used to remove anti-inflammatory mediators. In another embodiment, the sorbent is used to remove cytokines. In other embodiments, the sorbent may be used to remove checkpoint inhibitor proteins, or their soluble counterparts.

In other embodiments, when used in the immunomodulatory therapies, the sorbent may result in partial or full restoration of T-cell function.

Various non-limiting scenarios illustrate the treatments of the instant invention:
Scenario 1. Sorbent is used to treat immunoparalysis by removing factors that inhibit immune response to pathogens and other stimuli.
Scenario 2. Sorbent modulates the immune system during or after treatment of a subject with conventional supportive therapy methods.
Scenario 3. Sorbent is used to treat immunosenescence.
Scenario 4. Sorbent, combined with supportive therapy, improves overall effectiveness.

Certain embodiments of the invention are directed to methods of treating immunoparalysis in a subject in need thereof utilizing a porous biocompatible polymer sorbent of the disclosure. The methods of treating immunoparalysis may attenuate immunoparalysis. In certain embodiments, the methods reduce the severity of immunoparalysis and/or increase HLA-DR function. The methods also reduce the damage to the body caused by the hyperinflammatory response, thereby decreasing the patient's risk of immunoparalysis.

The methods treating immunoparalysis remove inflammatory (that can trigger apoptosis of immune effector cells and a rebound anti-inflammatory response) and anti-inflammatory mediators and/or checkpoint inhibitor proteins and their soluble counterparts from the patient. In other embodiments, the sorbent results in removal of one or more of a) cytokine and b) soluble ligand from bodily/physiologic fluid of the subject. In certain embodiments, the soluble ligand is one or more of PD-L1, PD-L2, sMICA, SULBP2, sFasL, sCTLA-4, MHC class II, and sCD40L. In one embodiment, the soluble ligand is PD-L1. In other embodiments, the cytokine comprises one or more of a member of interleukin, chemokine, interferon, lymphokine, tumor growth factor, or tumor necrosis factor family. In another embodiment, the sorbent reduces T-cell exhaustion.

In certain embodiments of the methods of treating immunoparalysis, the sorbent is used in conjunction with other supportive therapies. The sorbent may be provided/administered before, after, or concurrently with the supportive therapy. Similarly, the supportive therapy may be administered before, after, or concurrently with the supportive therapy.

In one embodiment, the method of treating immunoparalysis in a subject in need thereof includes contacting a physiologic fluid of the subject with a porous biocompatible polymer sorbent comprising a range of pore diameters between about 50 Å to about 3000 Å and a pore volume between about 0.5 cc/g to about 3.0 cc/g dry polymer.

In another embodiment, the method of treating immunoparalysis in a subject in need thereof includes contacting a physiologic fluid of the subject with a porous biocompatible polymer sorbent comprising a range of pore diameters between about 50 Å to about 40,000 Å and a pore volume between about 0.5 cc/g to about 5.0 cc/g dry polymer.

The method removes inflammatory mediators, and/or checkpoint proteins. In certain embodiments, wherein the contacting comprises administering the sorbent intravenously, intramuscularly, intratumorally, intradermally, intraperitoneally, subcutaneously, or orally, or nasally. In other embodiments, the sorbent is extracorporeal. In preferred embodiments, the physiologic fluid is blood.

In an embodiment, the method of treating immunoparalysis in a subject in need thereof includes administering a porous biocompatible polymer sorbent to the subject, wherein the polymer sorbent comprises a range of pore diameters between about 50 Å to about 3000 Å and a pore volume between about 0.5 cc/g to about 3.0 cc/g dry polymer, and wherein the administering places the sorbent in contact with a physiologic fluid of the subject. In preferred embodiments, the physiologic fluid is blood.

In another embodiment, the method of treating immunoparalysis in a subject in need thereof includes administering a porous biocompatible polymer sorbent to the subject, wherein the polymer sorbent comprises a range of pore diameters between about 50 Å to about 40,000 Å and a pore volume between about 0.5 cc/g to about 5.0 cc/g dry polymer, and wherein the administering places the sorbent in contact with a physiologic fluid of the subject. In preferred embodiments, the physiologic fluid is blood.

In an alternate embodiment, the method of treating immunoparalysis in a subject in need thereof includes providing to the subject a porous biocompatible polymer sorbent comprising a range of pore diameters between about 50 Å to about 3000 Å and a pore volume between about 0.5 cc/g to about 3.0 cc/g dry polymer. In an alternate embodiment, the method of treating immunoparalysis in a subject in need thereof includes providing to the subject a porous biocompatible polymer sorbent comprising a range of pore diameters between about 50 Å to about 40,000 Å and a pore volume between about 0.5 cc/g to about 5.0 cc/g dry polymer.

Another embodiment of the invention is use of a porous biocompatible polymer sorbent comprising a range of pore diameters between about 50 Å to about 3000 Å and a pore volume between about 0.5 cc/g to about 3.0 cc/g dry polymer for use in the treatment of immunoparalysis.

Another embodiment of the invention is use of a porous biocompatible polymer sorbent comprising a range of pore diameters between about 50 Å to about 40,000 Å and a pore volume between about 0.5 cc/g to about 5.0 cc/g dry polymer for use in the treatment of immunoparalysis.

An alternate embodiment of the invention is use of a porous biocompatible polymer sorbent comprising a range of pore diameters between about 50 Å to about 3000 Å and a pore volume between about 0.5 cc/g to about 3.0 cc/g dry polymer in manufacture of a medicament for treating immunoparalysis.

An alternate embodiment of the invention is use of a porous biocompatible polymer sorbent comprising a range of pore diameters between about 50 Å to about 40,000 Å and a pore volume between about 0.5 cc/g to about 5.0 cc/g dry polymer in manufacture of a medicament for treating immunoparalysis.

Any of the features described for the method of treating immunoparalysis are applicable to such uses.

Certain embodiments of the invention are directed to methods of treating immunosenescence in a subject in need thereof utilizing a porous biocompatible polymer sorbent of the disclosure. As with the treating immunoparalysis, the methods of treating immunosenescence, the methods of treating immunosenescence may result in removal of one or more of a) cytokine and b) soluble ligand from bodily/physiologic fluid of the subject. In certain embodiments, the soluble ligand is one or more of PD-L1, PD-L2, sMICA, SULBP2, sFasL, sCTLA-4, MHC class II, and sCD40L. In one embodiment, the soluble ligand is PD-L1. In other embodiments, the cytokine comprises one or more of a member of interleukin, chemokine, interferon, lymphokine, tumor growth factor, or tumor necrosis factor family.

In certain embodiments of the methods of treating immunosenescence, the sorbent is used in conjunction with other supportive therapies. The sorbent may be provided/administered before, after, or concurrently with the supportive therapy. Similarly, the supportive therapy may be administered before, after, or concurrently with the supportive therapy.

In one embodiment, the method of treating immunosenescence in a subject in need thereof includes contacting a physiologic fluid of the subject with a porous biocompatible polymer sorbent comprising a range of pore diameters between about 50 Å to about 3000 Å and a pore volume between about 0.5 cc/g to about 3.0 cc/g dry polymer.

In another embodiment, the method of treating immunosenescence in a subject in need thereof includes contacting a physiologic fluid of the subject with a porous biocompatible polymer sorbent comprising a range of pore diameters between about 50 Å to about 40,000 Å and a pore volume between about 0.5 cc/g to about 5.0 cc/g dry polymer.

The method removes inflammatory mediators, and/or checkpoint proteins. In certain embodiments, wherein the contacting comprises administering the sorbent intravenously, intramuscularly, intratumorally, intradermally, intraperitoneally, subcutaneously, or orally, or nasally. In other embodiments, the sorbent is extracorporeal. In preferred embodiments, the physiologic fluid is blood.

In another embodiment, the method of treating immunosenescence in a subject in need thereof includes administering a porous biocompatible polymer sorbent to the subject, wherein the polymer sorbent comprises a range of pore diameters between about 50 Å to about 3000 Å and a pore volume between about 0.5 cc/g to about 3.0 cc/g dry polymer, and wherein the administering places the sorbent in contact with a physiologic fluid of the subject. In preferred embodiments, the physiologic fluid is blood.

In another embodiment, the method of treating immunosenescence in a subject in need thereof includes administering a porous biocompatible polymer sorbent to the subject, wherein the polymer sorbent comprises a range of pore diameters between about 50 Å to about 40,000 Å and a pore volume between about 0.5 cc/g to about 5.0 cc/g dry polymer, and wherein the administering places the sorbent in contact with a physiologic fluid of the subject. In preferred embodiments, the physiologic fluid is blood.

In an alternate embodiment, the method of treating immunosenescence in a subject in need thereof includes providing to the subject a porous biocompatible polymer sorbent comprising a range of pore diameters between about 50 Å to about 3000 Å and a pore volume between about 0.5 cc/g to about 3.0 cc/g dry polymer.

In an alternate embodiment, the method of treating immunosenescence in a subject in need thereof includes providing to the subject a porous biocompatible polymer sorbent comprising a range of pore diameters between about 50 Å to about 40,000 Å and a pore volume between about 0.5 cc/g to about 5.0 cc/g dry polymer.

Another embodiment of the invention is use of a porous biocompatible polymer sorbent comprising a range of pore diameters between about 50 Å to about 3000 Å and a pore volume between about 0.5 cc/g to about 3.0 cc/g dry polymer for use in the treatment of immunosenescence.

Another embodiment of the invention is use of a porous biocompatible polymer sorbent comprising a range of pore diameters between about 50 Å to about 40,000 Å and a pore volume between about 0.5 cc/g to about 5.0 cc/g dry polymer for use in the treatment of immunosenescence.

An alternate embodiment of the invention is use of a porous biocompatible polymer sorbent comprising a range of pore diameters between about 50 Å to about 3000 Å and a pore volume between about 0.5 cc/g to about 3.0 cc/g dry polymer in manufacture of a medicament for treating immunosenescence.

An alternate embodiment of the invention is use of a porous biocompatible polymer sorbent comprising a range of pore diameters between about 50 Å to about 40,000 Å and a pore volume between about 0.5 cc/g to about 5.0 cc/g dry polymer in manufacture of a medicament for treating immunosenescence.

Any of the features described for the method of treating immunosenescence are applicable to such uses.

Certain embodiments of the invention are directed to methods of treating sepsis and/or inflammation related T-cell dysfunction in a subject in need thereof utilizing a porous biocompatible polymer sorbent of the disclosure. In one embodiment, the method treats sepsis. In another embodiment, the method treats inflammation related T-cell dysfunction. In an alternate embodiment, the method treats both sepsis and inflammation related T-cell dysfunction. In another embodiment, the method treats anti-inflammatory or immune suppression related T-cell dysfunction. In an alternative embodiment, the method treats both sepsis and anti-inflammatory or immune suppression related T-cell dysfunction.

In one embodiment of the invention, the method of treating sepsis and/or inflammation related T-cell dysfunction in a subject in need thereof includes contacting a physiologic fluid of the subject with a porous biocompatible polymer sorbent comprising a range of pore diameters between about 50 Å to about 3000 Å and a pore volume between about 0.5 cc/g to about 3.0 cc/g dry polymer.

In another embodiment of the invention, the method of treating sepsis and/or inflammation related T-cell dysfunction in a subject in need thereof includes contacting a physiologic fluid of the subject with a porous biocompatible polymer sorbent comprising a range of pore diameters between about 50 Å to about 40,000 Å and a pore volume between about 0.5 cc/g to about 5.0 cc/g dry polymer.

The method may remove inflammatory mediators, and/or checkpoint proteins. The method may also include administering the sorbent intravenously, intramuscularly, intratumorally, intradermally, intraperitoneally, subcutaneously, orally, rectally, topically, or nasally. Alternatively, the sorbent is extracorporeal. In preferred embodiments, the physiologic fluid is blood.

In an embodiment, the method of treating sepsis and/or inflammation related T-cell dysfunction in a subject in need thereof includes administering a porous biocompatible polymer sorbent to the subject, wherein the polymer sorbent comprises a range of pore diameters between about 50 Å to about 3000 Å and a pore volume between about 0.5 cc/g to about 3.0 cc/g dry polymer, and wherein the administering places the sorbent in contact with a physiologic fluid of the subject. In preferred embodiments, the physiologic fluid is blood.

In another embodiment, the method of treating sepsis and/or inflammation related T-cell dysfunction in a subject in need thereof includes administering a porous biocompatible polymer sorbent to the subject, wherein the polymer sorbent comprises a range of pore diameters between about 50 Å to about 40,000 Å and a pore volume between about 0.5 cc/g to about 5.0 cc/g dry polymer, and wherein the administering places the sorbent in contact with a physiologic fluid of the subject. In preferred embodiments, the physiologic fluid is blood.

In yet another embodiment, the method of treating sepsis and/or inflammation related T-cell dysfunction in a subject in need thereof comprising providing to the subject a porous biocompatible polymer sorbent comprising a range of pore diameters between about 50 Å to about 3000 Å and a pore volume between about 0.5 cc/g to about 3.0 cc/g dry polymer.

In yet another embodiment, the method of treating sepsis and/or inflammation related T-cell dysfunction in a subject in need thereof comprising providing to the subject a porous biocompatible polymer sorbent comprising a range of pore diameters between about 50 Å to about 40,000 Å and a pore volume between about 0.5 cc/g to about 5.0 cc/g dry polymer.

In certain embodiments of the methods of treating sepsis and/or inflammation related T-cell dysfunction, the sorbent is used in conjunction with other supportive therapies. The sorbent may be provided/administered before, after, or concurrently with the supportive therapy. Similarly, the supportive therapy may be administered before, after, or concurrently with the supportive therapy.

Another embodiment of the invention is use of a porous biocompatible polymer sorbent comprising a range of pore diameters between about 50 Å to about 3000 Å and a pore volume between about 0.5 cc/g to about 3.0 cc/g dry polymer for use in the treatment of sepsis and/or inflammation related T-cell dysfunction.

Another embodiment of the invention is use of a porous biocompatible polymer sorbent comprising a range of pore diameters between about 50 Å to about 40,000 Å and a pore volume between about 0.5 cc/g to about 5.0 cc/g dry polymer for use in the treatment of sepsis and/or inflammation related T-cell dysfunction.

An alternate embodiment of the invention is use of a porous biocompatible polymer sorbent comprising a range of pore diameters between about 50 Å to about 3000 Å and a pore volume between about 0.5 cc/g to about 3.0 cc/g dry polymer in manufacture of a medicament for treating sepsis and/or inflammation related T-cell dysfunction.

An alternate embodiment of the invention is use of a porous biocompatible polymer sorbent comprising a range of pore diameters between about 50 Å to about 40,000 Å and a pore volume between about 0.5 cc/g to about 5.0 cc/g dry polymer in manufacture of a medicament for treating sepsis and/or inflammation related T-cell dysfunction.

Any of the features described for the method of treating sepsis and/or inflammation related T-cell dysfunction are applicable to such uses.

In specific embodiments of the methods or uses, the sorbent sorbs PD-L1, PD-L2, or their soluble counterparts

In specific embodiments of the methods or uses, the sorbent sorbs anti-inflammatory cytokines.

As used herein, when the methods refer to administering, the administering encompasses giving the sorbent access to a physiologic fluid. For example, when the fluid is blood, access may be achieved by placing sorbent inside a blood vessel or by connecting to a blood vessel.

### EXAMPLES

The following examples are intended to be exemplary and non-limiting. The following examples of the invention are to further illustrate the nature of the invention. It is believed that one of ordinary skill in the art can, using the preceding description and the following illustrative examples, make and utilize the present invention and practice the claimed methods. It should be understood that the following examples do not limit the invention and that the scope of the invention is to be determined by the appended claims.

### Example 1: Base Sorbent Synthesis CY14175 & CY15077

Reactor Setup: a 4-neck glass lid was affixed to a 3 L jacketed cylindrical glass reaction vessel using a stainless-steel flange clamp and PFTE gasket. The lid was fitted with a PFTE stirrer bearing, RTD adapter, and water-cooled reflux condenser. A stainless-steel stirring shaft having five 60° agitators was fit through the stirrer bearing and inserted into a digital overhead stirrer. An RTD was fit through the corresponding adapter and connected to a Poly Stat circulating heating and chilling unit. Compatible tubing was used to connect the inlet and outlet of the reaction vessel jacket to the appropriate ports on the PolyStat. The unused port in the lid was used for charging the reactor and was plugged at all other times.

Polymerization: Aqueous phase and organic phase compositions are shown below, in Table I and Table II, respectively. Ultrapure water was split into approximately equal parts in two separate Erlenmeyer flasks, each containing a PFTE coated magnetic stir bar. Poly(vinyl alcohol) (PVA), having a degree of hydrolysis of 85.0 to 89.0 mol percent and a viscosity of 23.0 to 27.0 cP in a 4% aqueous solution at 20° C., was dispersed into the water in the first flask and heated to 80° C. on a hot plate with agitation. Salts (see Table 1, MSP, DSP, TSP and Sodium nitrite) were dispersed into the water in the second flask and heated to 80° C. on a hot plate with agitation. Circulation of heat transfer fluid from the PolyStat through the reaction vessel jacket was started, and fluid temperature heated to 60° C. Once PVA and salts dissolved, both solutions were charged to the reactor, one at a time, using a glass funnel. The digital overhead stirrer was powered on and the rpm set to a value to form appropriate droplet sizes upon organic phase addition. Temperature of the aqueous phase in the kettle was set to 70° C. The organic phase was prepared by adding benzoyl peroxide (BPO) to the divinylbenzene (DVB) in a 2 L Erlenmeyer flask and swirling until completely dissolved. 2,2,4-trimethylpentane and toluene were added to the flask, which was swirled to mix well. Once the temperature of the aqueous phase in the reactor reached 70° C., the organic phase was charged into the reactor using a narrow-necked glass funnel. Temperature of the reaction volume dropped upon the organic addition. A temperature program for the PolyStat was started, heating the reaction volume from 60 to 77° C. over 30 minutes, 77 to 80° C. over 30 minutes, holding the temperature at 80° C. for 960 minutes, and cooling to 20° C. over 60 minutes.

TABLE I
Aqueous Phase Composition

| Reagent | Mass (g) |
|---|---|
| Ultrapure water | 1500.000 |
| Poly(vinyl alcohol) (PVA) | 4.448 |
| Monosodium phosphate (MSP) | 4.602 |
| Disodium phosphate (DSP) | 15.339 |
| Trisodium phosphate (TSP) | 9.510 |
| Sodium nitrite | 0.046 |
| Total | 1533.899 |

TABLE II
Organic Phase Compositions

| | CY14175 | CY15077 |
|---|---|---|
| Reagent | Mass (g) | Mass (g) |
| Divinylbenzene, 63% (DVB) | 508.751 | 498.383 |
| 2,2,4-trimethylpentane (Isooctane) | 384.815 | 482.745 |
| Toluene | 335.004 | 222.404 |
| Benzoyl peroxide, 98% (BPO) | 3.816 | 3.738 |
| Total (excluding BPO) | 1228.571 | 1203.532 |

Work-up: reaction volume level in the reactor was marked. Overhead stirrer agitation was stopped, residual liquid siphoned out of the reactor, and the reactor filled to the mark with ultrapure water at room temperature. Overhead stirrer agitation was restarted, and the slurry heated to 70° C. as quickly as possible. After 30 minutes, agitation was stopped, and residual liquid siphoned out. Polymer beads were washed five times in this manner. During the final wash, the slurry temperature was cooled to room temperature. After the final water wash, polymer beads were washed with 99% isopropyl alcohol (IPA) in the same manner. 99% IPA was siphoned out and replaced with 70% IPA before transferring the slurry into a clean 4 L glass container. Unless noted otherwise, on an as-needed basis the polymer was steam stripped in a stainless steel tube for 8 hours, rewet in 70% IPA, transferred into DI water, sieved to obtain only the portion of beads having diameters between 300 and 600 μm, and dried at 100° C. until no further weight loss on drying was observed.

Cumulative pore volume data for polymers CY14175 and CY15077, measured by nitrogen desorption isotherm and mercury intrusion porosimetry, respectively, are shown below in Tables III and IV, respectively.

TABLE III
Nitrogen Desorption Isotherm Data for CY14175

| Pore Diameter | Pore size | Cumulative Pore |
|---|---|---|
| Range (Å) | Diameter (Å) | Volume (cm³/g) |
| 1411.9-1126.5 | 1236.810 | 0.018 |
| 1126.5-981.7 | 1043.980 | 0.038 |
| 981.7-752.9 | 836.783 | 0.142 |
| 752.9-659.9 | 700.102 | 0.243 |
| 659.9-572.0 | 609.466 | 0.417 |
| 572.0-483.1 | 519.809 | 0.646 |
| 483.1-449.8 | 465.223 | 0.730 |
| 449.8-401.4 | 422.725 | 0.849 |
| 401.4-354.1 | 374.629 | 0.956 |
| 354.1-337.9 | 345.602 | 0.997 |
| 337.9-313.5 | 324.759 | 1.055 |
| 313.5-290.8 | 301.243 | 1.097 |
| 290.8-262.8 | 275.230 | 1.1640 |
| 262.8-247.2 | 254.510 | 1.120 |
| 247.2-233.6 | 240.018 | 1.229 |
| 233.6-220.1 | 226.435 | 1.257 |
| 220.1-208.6 | 213.998 | 1.283 |
| 208.6-130.5 | 151.230 | 1.464 |
| 130.5-105.7 | 115.257 | 1.527 |
| 105.7-82.8 | 91.149 | 1.592 |
| 82.8-67.6 | 73.429 | 1.641 |
| 67.6-57.5 | 61.598 | 1.676 |
| 57.5-51.6 | 54.155 | 1.700 |
| 51.6-45.0 | 47.723 | 1.728 |
| 45.0-39.8 | 42.017 | 1.753 |
| 39.8-35.8 | 37.559 | 1.779 |
| 35.8-31.8 | 33.516 | 1.809 |
| 31.8-28.7 | 30.023 | 1.830 |
| 28.7-26.0 | 27.188 | 1.850 |
| 26.0-23.3 | 24.470 | 1.875 |
| 23.3-20.9 | 21.921 | 1.903 |
| 20.9-18.5 | 19.525 | 1.936 |
| 18.5-16.2 | 17.163 | 1.978 |

TABLE IV
Mercury Intrusion Data for CY15077

| Pore | Cumu- | Pore | Cumu- | Pore | Cumu- |
|---|---|---|---|---|---|
| size | lative | size | lative | size | lative |
| Diam- | Intru- | Diam- | Intru- | Diam- | Intru- |
| eter | sion | eter | sion | eter | sion |
| (Å) | (mL/g) | (A) | (mL/g) | (Å) | (mL/g) |
| 226299.063 | 3.401E−30 | 672.188 | 1.5811 | 111.948 | 2.1629 |
| 213166.078 | 0.0017 | 636.789 | 1.6027 | 108.883 | 2.1676 |
| 201295.156 | 0.0025 | 604.725 | 1.6218 | 106.648 | 2.1741 |
| 172635.813 | 0.0044 | 558.129 | 1.6515 | 104.522 | 2.1799 |
| 139538.063 | 0.0076 | 518.262 | 1.6789 | 102.430 | 2.1799 |
| 113120.781 | 0.0119 | 483.554 | 1.7086 | 100.158 | 2.1830 |
| 90542.367 | 0.0165 | 453.511 | 1.7359 | 98.293 | 2.1840 |
| 78733.258 | 0.0203 | 427.000 | 1.7559 | 96.448 | 2.1911 |
| 72446.375 | 0.0223 | 403.125 | 1.7836 | 94.422 | 2.1985 |
| 60340.402 | 0.0279 | 382.778 | 1.7938 | 91.526 | 2.2092 |
| 48343.840 | 0.0353 | 362.716 | 1.8178 | 89.258 | 2.2093 |
| 39009.137 | 0.0409 | 342.373 | 1.8388 | 87.078 | 2.2154 |
| 32136.408 | 0.0490 | 330.111 | 1.8515 | 85.424 | 2.2215 |
| 25330.656 | 0.0632 | 315.524 | 1.8697 | 83.626 | 2.2321 |
| 20981.516 | 0.0795 | 302.297 | 1.8851 | 82.112 | 2.2375 |
| 16219.864 | 0.1089 | 290.295 | 1.8951 | 79.916 | 2.2392 |
| 13252.412 | 0.1417 | 279.125 | 1.9124 | 78.015 | 2.2395 |
| 10501.536 | 0.1940 | 268.744 | 1.9243 | 76.200 | 2.2396 |
| 8359.911 | 0.2624 | 259.111 | 1.9360 | 75.092 | 2.2396 |
| 6786.301 | 0.3459 | 241.874 | 1.9551 | 73.412 | 2.2396 |
| 5538.123 | 0.4382 | 226.768 | 1.9730 | 72.237 | 2.2402 |
| 4337.931 | 0.5633 | 213.363 | 1.9881 | 71.100 | 2.2424 |
| 3501.675 | 0.6819 | 201.491 | 2.0075 | 69.863 | 2.2439 |
| 2838.742 | 0.8047 | 194.989 | 2.0221 | 68.408 | 2.2577 |
| 2593.017 | 0.8658 | 188.951 | 2.0339 | 67.137 | 2.2592 |
| 2266.689 | 0.9386 | 180.583 | 2.0351 | 66.034 | 2.2663 |
| 1831.042 | 1.0566 | 172.853 | 2.0507 | 65.082 | 2.2702 |
| 1509.851 | 1.1634 | 164.962 | 2.0629 | 64.044 | 2.2727 |
| 1394.006 | 1.2100 | 157.811 | 2.0711 | 62.385 | 2.2807 |
| 1294.780 | 1.2572 | 151.154 | 2.0821 | 61.328 | 2.2807 |
| 1207.693 | 1.2932 | 143.919 | 2.0965 | 60.304 | 2.2879 |
| 1131.861 | 1.3270 | 138.467 | 2.1069 | 59.414 | 2.2879 |
| 1065.100 | 1.3581 | 132.849 | 2.1193 | 58.547 | 2.2938 |
| 953.182 | 1.4059 | 129.576 | 2.1266 | 57.799 | 2.2976 |
| 884.036 | 1.4454 | 126.544 | 2.1266 | 56.890 | 2.2990 |
| 823.549 | 1.4787 | 124.2636 | 2.1323 | 55.921 | 2.3021 |
| 770.911 | 1.5106 | 120.898 | 2.1415 | 54.987 | 2.3034 |
| 722.472 | 1.5370 | 117.379 | 2.1508 | | |
| 684.612 | 1.5644 | 114.792 | 2.1548 | | |

### Example 2: Removal of Soluble PD-L1 (sPD-L1) from Bovine Whole Blood

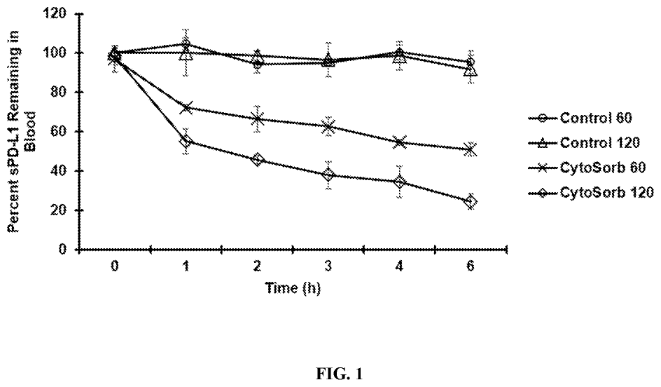

CytoSorb is a specific commercialized sorbent meeting the above polymer criteria that is manufactured by CytoSorbents. Data from bench top evaluations (FIG. 1) shows that CytoSorb removes soluble PD-L1 (sPD-L1) from bovine whole blood. In this study, two volumes of CytoSorb were evaluated: 60 mL and 120 mL. After 6 hours, CytoSorb 60 mL removed 49% of the starting sPD-L1 concentration, and CytoSorb 120 mL removed 76% of the starting sPD-L1 concentration. Controls (sham, no CytoSorb) were evaluated at the same time as each CytoSorb experiment. Each group was repeated 3 times (n=3).

### Example 3: Removal of Anti-Inflammatory Cytokines with Sorbent Therapy

Excessive anti-inflammatory cytokines can cause immune suppression. Removal of these substances through sorbent therapy has the potential to reverse immune suppression. Table V below summarizes the broad spectrum and robust in vitro removal of anti-inflammatory cytokines by CytoSorb.

TABLE V
% Removal of Anti-Inflammatory Mediators and Checkpoint
Inhibitors After Treatment with CytoSorb In Vitro

| | % Removed | Molecular | | |
|---|---|---|---|---|
| | After CS | Weight | Inflammatory | Length of |
| Analyte | Treatment | (kDa) | Status | Treatment |
| TNF-R2 | 63.17 | 20 | Anti | 2 hours |
| CD163 | 52.63 | 125 | Anti | 2 hours |
| IL-10 | 28.44 | 18.6 | Anti | 2 hours |
| IL-6Ra | 89.75 | 38 | Pro/Anti | 2 hours |
| gp130 | 85.36 | 68 | Pro/Anti | 2 hours |
| IL-19 | 100.00 | 18 | Pro/Anti | 2 hours |
| IFN-γ | 88.95 | 17 | Pro/Anti | 2 hours |
| IL-12 (p40) | 88.86 | 41 | Pro/Anti | 2 hours |
| IL-22 | 69.98 | 16.5 | Pro/Anti | 2 hours |
| IL-8 | 66.59 | 8 | Pro/Anti | 2 hours |
| IFN-α2 | 60.10 | 19.2 | Pro/Anti | 2 hours |
| TSLP | 52.71 | 15 | Pro/Anti | 2 hours |
| IL-10 | 79.00 | 18.6 | Anti | 6 hours |
| sPD-L1 | 49-75 | 31-41 | Immune | 6 hours |
| | | | Checkpoint | |

### Example 4: Removal of Inflammatory Cytokines and Pro-Inflammatory Mediators

As mentioned, the excessive production of pro-inflammatory cytokines and other inflammatory mediators can paradoxically lead to immune suppression by, for example, causing apoptosis of immune effector cells, causing upregulation of endothelial cell adhesion molecules that abnormally sequesters infection-fighting leukocytes to the endothelium of blood vessels in healthy organs and away from the site of infection, and a rebound anti-inflammatory response, also called the compensatory anti-inflammatory response syndrome (CARS). Sorbent treatment reduces these inflammatory mediators, which is intended to reduce the risk of these immunosuppressive complications.

The following data demonstrate the ability of CytoSorb, a commercialized sorbent therapy, to remove several prototypic pro-inflammatory cytokines, of which toxins play an important role.

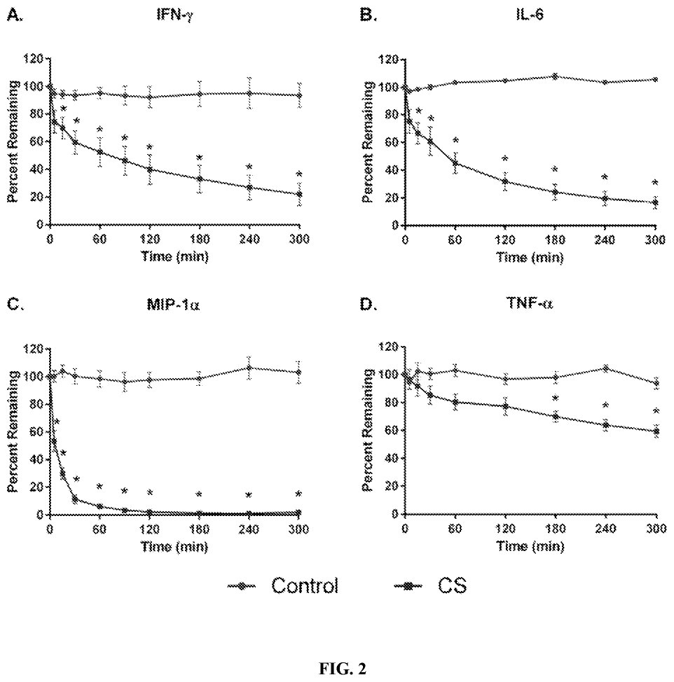

FIG. 2 demonstrates that the pro-inflammatory cytokines IL-6 (FIG. 2B), MCP-1α (FIG. 2C), tumor necrosis factor (TNF-α, (FIG. 2D) and interferon gamma (IFN-γ, FIG. 2A) are efficiently removed by CytoSorb.

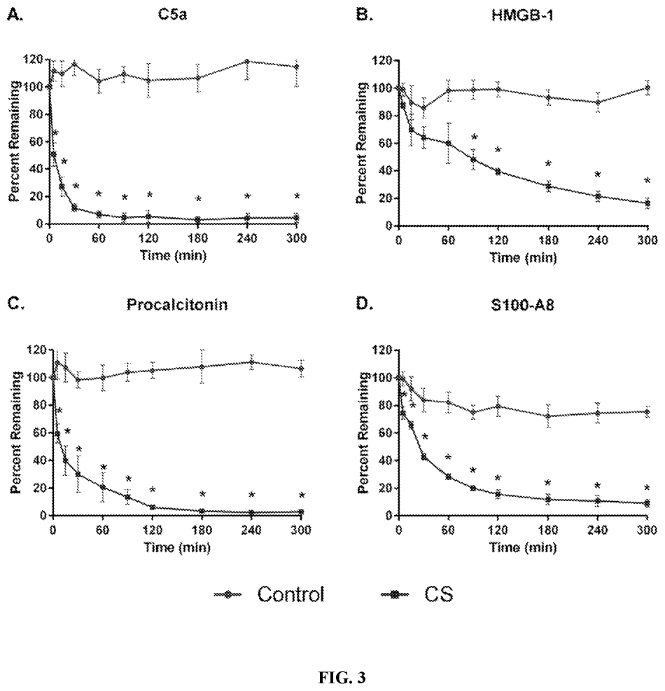

FIG. 3 demonstrates that inflammatory mediators such as damage associated molecular patterns (DAMPS) C5a (FIG. 3A), HMGB-1 (FIG. 3B), procalcitonin (FIG. 3C), and S100-A8 (FIG. 3D) are efficiently removed by CytoSorb.

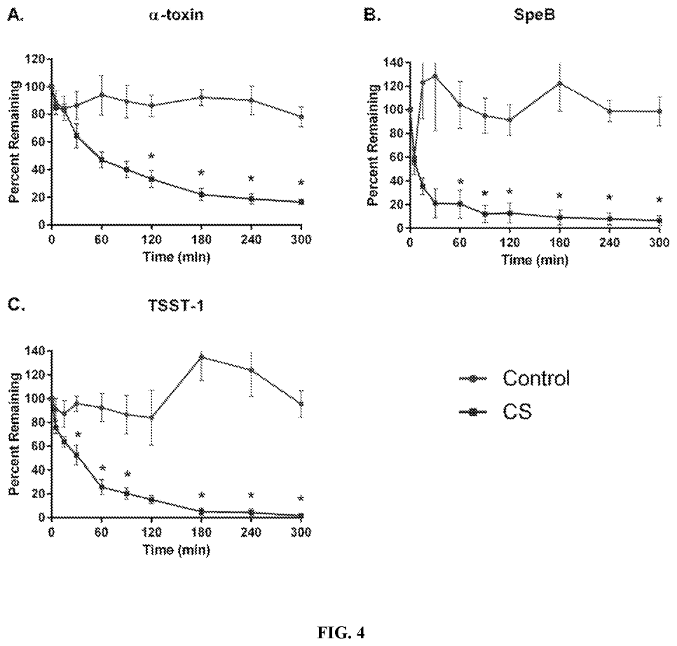

FIG. 4 demonstrates that inflammatory mediators such as pattern associated molecular patterns (PAMPS) α-toxin (FIG. 4A), SpeB (FIG. 4B), and TSST-1 (FIG. 4C) are efficiently removed by CytoSorb.

These experiments were done in vitro by adding purified inflammatory proteins into a 3.8% citrated whole blood recirculation system, and pumping the blood through either a cartridge containing the CytoSorb polymer (13:3 v/v blood to polymer) covered by the specifications above) in blue, or a sham (no polymer) cartridge in red at flow rates ranging from 20-140 mL/min. The rapid reduction of these inflammatory mediators can be seen in the graphs below. In the human treatment of immunosuppression by CytoSorb, CytoSorb would be used in an extracorporeal CRRT circuit at flow rates of 150-400 mL/min, continuously, changing the device to a new CytoSorb device every 8 to 24 hours, where the reduction of these inflammatory mediators and improved immune responsiveness is expected.

Reference: Gruda, et al. PLOS ONE 2018; 13(1):e0191676.

### Example 5: Evaluation of Immune System Function Before and After Sorbent Treatment

To demonstrate the effect of sorbent treatment on immune suppression or immunoparalysis, blood samples are taken from the patient prior to, and then after, sorbent treatment and compared for a number of parameters.

The ImmuKnow® test (Eurofins Viracor; Missouri, USA) is a commercially available assay that measures ATP production in response to PHA stimulation of isolated CD4+ T cells from peripheral blood using an ATP luciferase assay. An Immuknow level ≤225 ng/ml indicates a low immune cell response, a level of 226-524 mg/mL is a moderate immune response, and ≥525 ng/ml represents a high immune cell response. [http://portals.clevelandclinic.org/portals/66/PDF/TechBriefs/TB_ImmuneFunctionAssay.pdf]

The effect of CytoSorb on immune suppression as measured by the Immuknow test is exemplified by two septic patients treated with CytoSorb in an extracorporeal circuit with CRRT for 6 hours a day for 7 consecutive days. The data is summarized below in Table VI. The ImmuKnow assay was performed prior to the first treatment on Day 1 and then on Day 3, 5, and 7. The first patient was a 59 year old man with pneumonia, renal failure, and septic shock. The second patient was a 72 year old woman with aspiration pneumonia and septic shock.

TABLE VI
ImmuKnow Values (ng/mL)

| | | | | | | | |
|---|---|---|---|---|---|---|---|
| CytoSorb Treatment | Day 1 | Day 2 | Day 3 | Day 4 | Day 5 | Day 6 | Day 7 |

| | | | | |
|---|---|---|---|---|
| Patient #1 | 113 | 255 | 405 | 582 |
| Patient #2 | 185 | 846 | >1,000 | 757 |

The initial ImmuKnow values in each of these patients on Day 1 prior to CytoSorb treatment demonstrate a low immune response and immune suppression. With successive CytoSorb treatments, the ImmuKnow values rose, indicating greater responsiveness of CD4+ T cells to PHA stimulation, and a reversal of immune suppression.

### Example 6: Stimulation of Immune Cell Activation Following Sorbent Treatment of Patient or Patient's Blood

During immunosuppression, upregulation of key signaling molecules including the anti-inflammatory cytokines TGF-β and IL-10 can lead to increased proliferation of regulatory T cells (T_{regs}) via transdifferentiation of T_{H}1 helper T cells or de-novo differentiation from naïve T cells [Battaglia 06, Gutcher 07]. Regulatory T cells function to downregulate the activity of both proximal and distal effector T cells through direct cell to cell contact inhibition and secreted cytokines (e.g. IL-10 and TGF-β) [Gregori 12]. IL-10 directly inhibits the production of proinflammatory cytokines IL-2, IFNγ, and GM-CSF by effector T cells, thus shifting the adaptive immune response from an active state to a suppressive state [Yudoh 00].

The potential therapy application proposed in this patent is the direct removal of excess levels of anti-inflammatory cytokines and other soluble, secreted immunosuppressive factors in patients that are severely immunosuppressed as a result of cytokine storm arising from various diseases including sepsis. The action of the therapy can be assessed in a humanized in vivo animal model of immunosuppression by quantifying the proportion of circulating activated effector T cells versus regulatory T cells [Jespersen 17, Wu 13]. Immunosuppressed individuals usually will have a higher abundance of regulatory T cells and fewer activated effector cells as a result. The different T-cell lineages can be mapped by antibody staining of unique surface or intracellular markers present on each T-cell type and enumerated via flow cytometry and fluorescence assisted cell sorting (FACS) using a gating approach. All T cells can be identified from the other leukocyte classes by staining for CD3, a component of the surface T-cell receptor. A secondary stain specific for surface CD4 and CD8 can be applied to distinguish CD4⁺ helper T cells from CD8⁺ cytotoxic T cells. Finally, a tertiary stain can be applied against surface CD69, CD25, HLA-DR, and CD122, for example, to differentiate activated T cells from nonactivated cells; a stain against surface CD152 (CTLA4) and intracellular Foxp3 can be included to identify the regulatory T cells [Bajnok 17, Mousset 19]. It is expected that following treatment, there will be a reduction in the systemic levels of key anti-inflammatory immunosuppressive factors, resulting in a marked decrease in the proliferation and activity of regulatory T cells, ultimately alleviating suppression of effector T cell activation and allowing for increased proliferation of these effector cells. This phenomenon will be easily visualized using the method outlined above, whereby the flow cytometry gating output will reveal an obvious shift in the T cell populations to a greater abundance of activated effecter T cells and fewer regulatory T cells after treatment.

### REFERENCES

Gregori, Silvia, Kevin S. Goudy, and Maria Grazia Roncarolo. “The cellular and molecular mechanisms of immuno-suppression by human type 1 regulatory T cells.” Frontiers in immunology 3 (2012): 30.
Yudoh, Kazuo, et al. “Reduced expression of the regulatory CD4+ T cell subset is related to Th1/Th2 balance and disease severity in rheumatoid arthritis.” Arthritis & Rheumatism: Official Journal of the American College of Rheumatology 43.3 (2000): 617-627.
Battaglia, Manuela, et al. “Tr1 cells: from discovery to their clinical application.” Seminars in immunology. Vol. 18. No. 2. Academic Press, 2006.
Gutcher, Ilona, and Burkhard Becher. “APC-derived cytokines and T cell polarization in autoimmune inflammation.” The Journal of clinical investigation 117.5 (2007): 1119-1127.
Mousset, Charlotte M., et al. “Comprehensive phenotyping of T cells using flow cytometry.” Cytometry Part A 95.6 (2019): 647-654.
Bajnok, Anna, et al. “The distribution of activation markers and selectins on peripheral T lymphocytes in preeclampsia.” Mediators of inflammation 2017 (2017).
Jespersen, Henrik, et al. “Clinical responses to adoptive T-cell transfer can be modeled in an autologous immune-humanized mouse model.” Nature communications 8.1 (2017): 1-10.
Wu, Douglas C., et al. “Ex vivo expanded human regulatory T cells can prolong survival of a human islet allograft in a humanized mouse model.” Transplantation 96.8 (2013): 707.

### Example 7: Other Methods of Measuring Immune Cell Function and Reversal of Immune Suppression or Before and After Sorbent Treatment

Number of leukocytes as measured by complete blood count and white blood cell differential is a crude measure of immune function and immune cell depletion. In patients who are neutropenic, sorbent treatment may reduce immune suppression, and promote cell growth and differentiation, resulting in an increase or repopulation of immune cells.

Activation level of peripheral blood mononuclear cells (PBMCs such as T-cells, B-cells, natural killer cells, monocytes) are assessed by fluorescence activated cell sorting (FACS) based on cell-surface markers of cell activation such as HLA-DR (see Example 6). Intracellular metabolic activity can also be detected by FACS. For example, exposure of activated neutrophils with phorbol myristate acetate (PMA) results in oxidative bursts (e.g. hydrogen peroxide) via NADPH oxidase activity that can be detected by FACS using dihydrorhodamine. Following sorbent treatment, FACS analysis is expected to demonstrate a higher proportion of activated PBMCs or neutrophils.

Immune function assays also evaluate the ability of purified PBMCs or those in whole blood to respond to various stimuli. One example is the cell proliferation assay where PBMCs are exposed an activator such as phytohemagglutinin (PHA), pokeweed mitogen (PWM), or tetanus toxoid. For lymphocytes, this assay is also called the lymphocyte proliferation or lymphocyte transformation test. Immunoparalyzed cells will either not proliferate or proliferate slowly, where activated cells will proliferate more quickly and increase in cell numbers. Other methods of detecting cell proliferation include, but are not limited to cell cycle analysis, DNA histogramming, co-culture cytotoxicity and other techniques to detect the percentage of replicating or functional cells. Sorbent treatment is expected to increase this percentage. In another example, PBMCs are stimulated with an activator that increases transcription or metabolic activity, including PHA as in Example 4, or substances such as lipopolysaccharide endotoxin. In the latter, anergic This includes PHA, as in Example 5, and other activators. For example, stimulation with endotoxin in normal or activated immune cells will result in these cells producing cytokines that can be immunoassayed. The production of cytokines will be low in quiescent or immune suppressed cells and increase during and after sorbent treatment. An example of sorbent treatment is when blood is recirculated out of the body, through a temporary dialysis catheter in a major vein, through a sorbent cartridge, and back into the body repeatedly.

### Example 8: Methods of Reducing Functional Immunosuppression by Reducing Abnormal Sequestration of Leukocytes and Improving Leukocyte Targeting to an Area of Injury and Infection

Cell-mediated immunity is important in the response to injury and infection. For example, a known genetic disorder called Leukocyte adhesion deficiency type I that prevents leukocytes from binding to the blood vessel wall and penetrating into the area of infection results in a functional immune suppression and a high susceptibility to life-threatening bacterial and fungal infections.

In sepsis, hyperinflammation and the overexpression of cytokines, called cytokine storm, can lead to a global upregulation of endothelial cell adhesion molecules, and leukocyte adhesion molecules and integrins that result in the unwanted binding of leukocytes to the endothelium of blood vessels throughout the body. This abnormal sequestration of leukocytes decreases the availability of these immune cells to fight injury or infection at the true site of infection or injury. This process also interferes with the normal ability of leukocytes to locate the true site of infection or injury via the normal process of leukocyte rolling, adhesion, and transendothelial migration. Even though the leukocytes are activated, the inability to get to the site of injury or infection results in a functional immune suppression.

For example, using sorbent therapy in an extracorporeal circuit to reduce circulating inflammatory cytokines and mediators in sepsis, for example, we would expect to observe a partial or full resolution of this functional immune suppression with decreased global expression of endothelial cell adhesion molecules, decreased abnormal margination of leukocytes, increased numbers of leukocytes and decreased bacterial counts (or improved source control) at the site of infection, and decreased leukocyte infiltration and cell mediated damage in uninfected organs and tissues.

### Aspects of the Disclosure

A first aspect of the invention relates to a method of treating immune suppression, anergy, or immunoparalysis by the attenuation via removal of inflammatory, and/or anti-inflammatory mediators, and/or checkpoint inhibitor proteins in a subject, comprising treating a subject with a therapeutically effective amount of porous biocompatible polymer sorbent.

A second aspect of the invention relates to the method according to the first aspect, wherein the sorbent comprises a range of pore diameters between about 50 Å to about 3000 Å and a pore volume between about 0.5 cc/g to about 3 cc/g dry polymer.

A third aspect of the invention relates to the method according to the first aspect, wherein the sorbent comprises a range of pore diameters between about 50 Å to about 40,000 Å and a pore volume between about 0.5 cc/g to about 5 cc/g dry polymer.

A fourth aspect of the invention relates to the method according to the any one of the first-third aspects, wherein the sorbent is administered extracorporeally.

A fifth aspect of the invention relates to the method according to the any one of the first-third aspects, wherein the sorbent is administered intravenously, intramuscularly, intratumorally, intradermally, intraperitoneally, subcutaneously, orally, rectally, topically, or nasally.

A sixth aspect of the invention relates to the method according to the third aspect, wherein the ratio of pore volume between 50 Å to 40,000 Å (pore diameter) to pore volume between 1,000 Å to 10,000 Å (pore diameter) of the sorbent is smaller than 2:1.

A seventh aspect of the invention relates to the method according to any of the preceding aspects, wherein the sorbent is produced using at least one crosslinking agent and at least one monomer.

An eight aspect of the invention relates to the method according to the seventh aspect, wherein the monomer comprises one or more of divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, and divinylformamide.

A ninth aspect of the invention relates to the method according to the seventh aspect or eight aspect, wherein the crosslinking agent comprises one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

A tenth aspect of the invention relates to the method according to the any one of the seventh-ninth aspects, wherein the sorbent is produced additionally utilizing one or both of at least one dispersing agent and at least one porogen.

An eleventh aspect of the invention relates to the method according to the any one of the first-tenth aspects, wherein the sorbent comprises a biocompatible and hemocompatible exterior coating that is covalently bound to the sorbent by free-radical grafting.

A twelfth aspect of the invention relates to the method according to the any one of the first-eleventh aspects, wherein said sorbent is administered prior to administration of supportive therapies.

A thirteenth aspect of the invention relates to the method according to the any one of the first-eleventh aspects, wherein said sorbent is administered at the same time as administration of supportive therapies.

A fourteenth aspect of the invention relates to the method according to the any one of the first-eleventh aspects, wherein said sorbent is administered following administration of supportive therapies.

A fifthteenth aspect of the invention relates to the method according to the any one of the twelfth-fourteenth aspects, wherein the supportive therapy comprises administration of an immunotherapeutic agent selected from one or more of checkpoint inhibitors, monoclonal antibodies, and bi-specific T-cell engagers.

A sixteenth aspect of the invention relates to the method according to the any one of the first-eleventh aspects, wherein the administration of the sorbent reduces the severity of immunoparalysis.

A seventeenth aspect of the invention relates to the method according to the any one of the first-eleventh aspects, wherein the administration of the sorbent increases HLA-DR expression or function.

An eighteenth aspect of the invention relates to the method according to the any one of the first-eleventh aspects, wherein the administration of the sorbent reduces the damage to the body caused by the hyperinflammatory response, thereby decreasing the patient's risk of immunoparalysis.

A nineteenth aspect of the invention relates to the method according to the any one of the first-eleventh aspects, wherein administration of the sorbent reduces T-cell or other immune cell exhaustion.

A twentieth aspect of the invention relates to the method according to the any one of the first-eleventh aspects, wherein administration of the sorbent results in removal of one or more of a) cytokine and b) soluble ligand from bodily/physiologic fluid of the subject.

A twenty-first aspect of the invention relates to the method according to the any one of the first-eleventh aspects, wherein administration of the sorbent results in removal of inflammatory mediators.

A twenty-second aspect of the invention relates to the method according to the any one of the first-eleventh aspects, wherein administration of the sorbent results in removal of one or more checkpoint inhibitor proteins and/or their soluble counterparts.

A twenty-third aspect of the invention relates to the method according to the any one of the first-eleventh aspects, wherein administration of the sorbent results in partial or full restoration of T-cell or other immune cell function.

A twenty-fourth aspect of the invention relates to the method according to the twentieth aspect, wherein the soluble ligand is one or more of sPD-L1, sPD-L2, sMICA, SULBP2, sFasL, sCTLA-4, MHC class II, and sCD40L.

A twenty-fifth aspect of the invention relates to the method according to the twentieth aspect, wherein the cytokine comprises one or more of a member of interleukin, chemokine, interferon, lymphokine, tumor growth factor, or tumor necrosis factor family.

A twenty-sixth aspect of the invention relates to the method according to the any one of the first-twenty-fifth aspects, wherein the sorbent comprises one or more residues of divinylbenzene and ethylvinylbenzene, styrene, and ethylstyrene monomers.

A twenty-seventh aspect of the invention relates to a method of reducing sepsis and inflammation related T-cell dysfunction in a subject comprising treating the subject with a therapeutically effective amount of porous biocompatible polymer sorbent.

A twenty-eighth aspect of the invention relates to the method according to the twenty-seventh aspect, wherein the sorbent comprises a range of pore diameters between about 50 Å to about 3000 Å and a pore volume between about 0.5 cc/g to about 3 cc/g dry polymer.

A twenty-ninth aspect of the invention relates to the method according to the twenty-seventh aspect, wherein the sorbent comprises a range of pore diameters between about 50 Å to about 40,000 Å and a pore volume between about 0.5 cc/g to about 5 cc/g dry polymer.

A thirtieth aspect of the invention relates to the method according to the any one of the twenty-seventh-twenty-ninth aspects, wherein the sorbent is administered extracorporeally.

A thirty-first aspect of the invention relates to the method according to the any one of the twenty-seventh-twenty-ninth aspects, wherein the sorbent is administered intravenously, intramuscularly, intratumorally, intradermally, intraperitoneally, subcutaneously, orally, rectally, topically, or nasally.

A thirty-second aspect of the invention relates to the method according to the twenty-ninth aspect, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

A thirty-third aspect of the invention relates to the method according to the any one of the twenty-seventh-thirty-second aspects, wherein the sorbent is produced using at least one crosslinking agent and at least one monomer.

A thirty-fourth aspect of the invention relates to the method according to the thirty-third aspect, wherein the monomer comprises divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

A thirty-fifth aspect of the invention relates to the method according to the thirty-third aspect or thirty-fourth aspect, wherein the crosslinking agent comprises one or more or divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

A thirty-sixth aspect of the invention relates to a method of treating immunoparalysis in a subject in need thereof comprising contacting a physiologic fluid of the subject with a porous biocompatible polymer sorbent, wherein method removes inflammatory mediators, and/or anti-inflammatory mediators, and/or checkpoint proteins.

A thirty-seventh aspect of the invention relates to the method according to the thirty-sixth aspect, wherein the sorbent comprises a range of pore diameters between about 50 Å to about 3000 Å and a pore volume between about 0.5 cc/g to about 3 cc/g dry polymer.

A thirty-eighth aspect of the invention relates to the method according to the thirty-sixth aspect, wherein the sorbent comprises a range of pore diameters between about 50 Å to about 40,000 Å and a pore volume between about 0.5 cc/g to about 5 cc/g dry polymer.

A thirty-ninth aspect of the invention relates to the method according to the any one of the thirty-sixth-thirty-eighth aspects, wherein the contacting comprises administering the sorbent intravenously, intramuscularly, intratumorally, intradermally, intraperitoneally, subcutaneously, orally, rectally, topically, or nasally.

A fortieth aspect of the invention relates to the method according to the any one of the thirty-sixth-thirty-eighth aspects, wherein the contacting comprises administering the sorbent extracorporeally.

A forty-first aspect of the invention relates to a method of treating immunoparalysis in a subject in need thereof comprising administering a porous biocompatible polymer sorbent to the subject, wherein the administering places the sorbent in contact with a physiologic fluid of the subject.

A forty-second aspect of the invention relates to the method according to the forty-first aspect, wherein the sorbent comprises a range of pore diameters between about 50 Å to about 3000 Å and a pore volume between about 0.5 cc/g to about 3 cc/g dry polymer.

A forty-third aspect of the invention relates to the method according to the forty-first aspect, wherein the sorbent comprises a range of pore diameters between about 50 Å to about 40,000 Å and a pore volume between about 0.5 cc/g to about 5 cc/g dry polymer.

A forty-fourth aspect of the invention relates to the method according to any one of the forty-first aspect-forty-third aspect, wherein the physiologic fluid is blood.

A forty-fifth aspect of the invention relates to the method according to any one of the forty-first aspect-forty-fourth aspect, wherein the sorbent is administered extracorporeally.

A forty-sixth aspect of the invention relates to the method according to any one of the forty-first aspect-forty-fourth aspect, wherein the sorbent is administered intravenously, intramuscularly, intratumorally, intradermally, intraperitoneally, subcutaneously, orally, rectally, topically, or nasally.

A forty-seventh aspect of the invention relates to a method of treating immunoparalysis in a subject in need thereof comprising providing to the subject a porous biocompatible polymer sorbent.

A forty-eighth aspect of the invention relates to the method according to the forty-seventh aspect, wherein the sorbent comprises a range of pore diameters between about 50 Å to about 3000 Å and a pore volume between about 0.5 cc/g to about 3 cc/g dry polymer.

A forty-ninth aspect of the invention relates to the method according to the forty-seventh aspect, wherein the sorbent comprises a range of pore diameters between about 50 Å to about 40,000 Å and a pore volume between about 0.5 cc/g to about 5 cc/g dry polymer.

A fiftieth aspect of the invention relates to the method according to any one of the forty-seventh aspect-forty-ninth aspects, wherein the administration of the sorbent reduces the severity of immunoparalysis, immune suppression, or anergy.

A fifty-first aspect of the invention relates to the method according to any one of the forty-seventh aspect-forty-ninth aspects, wherein the administration of the sorbent reduces the damage to the body caused by the hyperinflammatory response, thereby decreasing the patient's risk of immunoparalysis, immune suppression, or anergy.

A fifty-second aspect of the invention relates to the method according to any one of the forty-seventh aspect-forty-ninth aspects, wherein administration of the sorbent reduces T-cell exhaustion.

A fifty-third aspect of the invention relates to the method according to any one of the forty-seventh aspect-forty-ninth aspects, wherein the sorbent results in removal of one or more of a) cytokine and b) soluble ligand from bodily/physiologic fluid of the subject.

A fifty-fourth aspect of the invention relates to the method according to any one of the forty-seventh aspect-forty-ninth aspects, wherein the sorbent results in removal of inflammatory mediators.

A fifty-fifth aspect of the invention relates to the method according to any one of the forty-seventh aspect-forty-ninth aspects, wherein the sorbent results in removal of checkpoint inhibitor proteins.

A fifty-sixth aspect of the invention relates to the method according to any one of the forty-seventh aspect-forty-ninth aspects, wherein the sorbent results in results in partial or full restoration of T-cell function.

A fifty-seventh aspect of the invention relates to the method according to the fifty-third aspect, wherein the soluble ligand is one or more of sPD-L1, sPD-L2, sMICA, SULBP2, sFasL, sCTLA-4, MHC class II, and sCD40L.

A fifty-eighth aspect of the invention relates to the method according to the fifty-third aspect, wherein the cytokine comprises one or more of a member of interleukin, chemokine, interferon, lymphokine, tumor growth factor, or tumor necrosis factor family.

A fifty-ninth aspect of the invention relates to the method according to any one of the forty-seventh aspect-fifty-eighth aspects, wherein the sorbent is administered extracorporeally.

A sixtieth aspect of the invention relates to the method according to any one of the forty-seventh aspect-fifty-eighth aspects, wherein the sorbent is administered intravenously, intramuscularly, intratumorally, intradermally, intraperitoneally, subcutaneously, orally, rectally, topically, or nasally.

A sixty-first aspect of the invention relates to a method of treating sepsis and/or inflammation related T-cell or immune cell dysfunction in a subject in need thereof comprising contacting a physiologic fluid of the subject with a porous biocompatible polymer sorbent.

A sixty-second aspect of the invention relates to the method according to the sixty-first aspect, wherein the sorbent comprises a range of pore diameters between about 50 Å to about 3000 Å and a pore volume between about 0.5 cc/g to about 3 cc/g dry polymer.

A sixty-third aspect of the invention relates to the method according to the sixty-first aspect, wherein the sorbent comprises a range of pore diameters between about 50 Å to about 40,000 Å and a pore volume between about 0.5 cc/g to about 5 cc/g dry polymer.

A sixty-fourth aspect of the invention relates to the method according to any one of the sixty-first aspects-sixty-third aspects, wherein the contacting comprises administering the sorbent intravenously, intramuscularly, intratumorally, intradermally, intraperitoneally, subcutaneously, orally, rectally, topically, or nasally.

A sixty-fifth aspect of the invention relates to the method according to any one of the sixty-first aspects-sixty-third aspects, wherein the contacting comprises administering the sorbent extracorporeally.

A sixty-sixth aspect of the invention relates to a method of treating sepsis and/or inflammation related T-cell or other immune cell dysfunction in a subject in need thereof comprising administering a porous biocompatible polymer sorbent to the subject, wherein the administering places the sorbent in contact with a physiologic fluid of the subject.

A sixty-seventh aspect of the invention relates to the method according to any one of the forty-seventh aspects-sixty-sixth aspects, wherein the physiologic fluid is blood.

A sixty-eighth aspect of the invention relates to a method of treating sepsis and/or inflammation related T-cell dysfunction in a subject in need thereof comprising providing to the subject a porous biocompatible polymer sorbent.

A sixty-ninth aspect of the invention relates to the method according to any one of the sixty-sixth aspects-sixty-eighth aspects, wherein the sorbent comprises a range of pore diameters between about 50 Å to about 3000 Å and a pore volume between about 0.5 cc/g to about 3 cc/g dry polymer.

A seventieth aspect of the invention relates to the method according to any one of the sixty-sixth aspects-sixty-eighth aspects, wherein the sorbent comprises a range of pore diameters between about 50 Å to about 40,000 Å and a pore volume between about 0.5 cc/g to about 5 cc/g dry polymer.

A seventy-first aspect of the invention relates to the method according to any one of the sixty-sixth aspects-sixty-eighth aspects, wherein the contacting comprises administering the sorbent intravenously, intramuscularly, intratumorally, intradermally, intraperitoneally, subcutaneously, orally, rectally, topically, or nasally.

A seventy-second aspect of the invention relates to the method according to any one of the sixty-sixth aspects-sixty-eighth aspects, wherein the contacting comprises administering the sorbent extracorporeally.

A seventy-third aspect of the invention relates to the method according to the seventieth aspect, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

A seventy-fourth aspect of the invention relates to the method according to any one of the sixty-sixth aspects-seventy-third aspects, wherein the sorbent is produced using at least one crosslinking agent and at least one monomer.

A seventy-fifth aspect of the invention relates to the method according to the seventy-fourth aspect, wherein the monomer comprises one or more of divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, and divinylformamide.

A seventy-sixth aspect of the invention relates to the method according to the seventy-fourth aspect or seventy-fifth aspect, wherein the crosslinking agent comprises one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

A seventy-seventh aspect of the invention relates to the method according to the seventy-fourth aspect or seventy-fifth aspect, wherein the sorbent is produced additionally utilizing one or both of at least one dispersing agent and at least one porogen.

A seventy-eighth aspect of the invention relates to the method according to any one of the sixty-sixth aspects-seventy-third aspects, wherein the sorbent comprises a biocompatible and hemocompatible exterior coating that is covalently bound to the sorbent by free-radical grafting.

A seventy-ninth aspect of the invention relates to the method according to any one of the sixty-sixth aspects-seventy-third aspects, wherein said sorbent is administered prior to administration of supportive therapies.

An eightieth aspect of the invention relates to the method according to any one of the sixty-sixth aspects-seventy-third aspects, wherein said sorbent is administered at the same time as administration of supportive therapies.

An eighty-first aspect of the invention relates to the method according to any one of the sixty-sixth aspects-seventy-third aspects, wherein said sorbent is administered following administration of supportive therapies.

An eighty-second aspect of the invention relates to the method according to the eighty-first aspect, wherein the supportive therapy comprises administration of an immunotherapeutic agent selected from one or more of checkpoint inhibitors, monoclonal antibodies, and bi-specific T-cell engagers.

An eighty-third aspect of the invention relates to the method according to any one of the sixty-sixth aspects-seventy-third aspects, wherein the sorbent comprises one or more residues of divinylbenzene and ethylvinylbenzene, styrene, and ethylstyrene monomers.

An eighty-fourth aspect of the invention relates to the method according to any one of the first aspects-twenty-sixth aspects, wherein administration for the sorbent results in decreased endothelial sequestration of leukocytes and/or increased presence of leukocytes to the area of infection or injury, and/or reduction in infection and/or improved healing.

What is claimed:

1. A method of treating immune suppression, anergy, or immunoparalysis by the attenuation via removal of inflammatory, and/or anti-inflammatory mediators, and/or checkpoint inhibitor proteins in a subject, comprising treating a subject with a therapeutically effective amount of porous biocompatible polymer sorbent.

2. The method of claim 1, wherein the sorbent comprises a range of pore diameters between about 50 Å to about 3000 Å and a pore volume between about 0.5 cc/g to about 3 cc/g dry polymer.

3. The method of claim 1, wherein the sorbent comprises a range of pore diameters between about 50 Å to about 40,000 Å and a pore volume between about 0.5 cc/g to about 5 cc/g dry polymer.

4. The method of any one of claims 1-3, wherein the sorbent is administered extracorporeally.

5. The method of any one of claims 1-3, wherein the sorbent is administered intravenously, intramuscularly, intratumorally, intradermally, intraperitoneally, subcutaneously, orally, rectally, topically, or nasally.

6. The method of claim 3, wherein the ratio of pore volume between 50 Å to 40,000 Å (pore diameter) to pore volume between 1,000 Å to 10,000 Å (pore diameter) of the sorbent is smaller than 2:1.

7. The method of any one of the preceding claims, wherein the sorbent is produced using at least one crosslinking agent and at least one monomer.

8. The method of claim 7, wherein the monomer comprises one or more of divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, and divinylformamide.

9. The method of claim 7 or claim 8, wherein the crosslinking agent comprises one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

10. The method of any one of claims 7-9, wherein the sorbent is produced additionally utilizing one or both of at least one dispersing agent and at least one porogen.

11. The method of any one of claims 1-10, wherein the sorbent comprises a biocompatible and hemocompatible exterior coating that is covalently bound to the sorbent by free-radical grafting.

12. The method of any one of claims 1-11, wherein said sorbent is administered prior to administration of supportive therapies.

13. The method of any one of claims 1-11, wherein said sorbent is administered at the same time as administration of supportive therapies.

14. The method of any one of claims 1-11, wherein said sorbent is administered following administration of supportive therapies.

15. The method of anyone of claims 12-14, wherein the supportive therapy comprises administration of an immunotherapeutic agent selected from one or more of checkpoint inhibitors, monoclonal antibodies, and bi-specific T-cell engagers.

16. The method of any one of claims 1-11, wherein the administration of the sorbent reduces the severity of immunoparalysis.

17. The method of any one of claims 1-11, wherein the administration of the sorbent increases HLA-DR expression or function.

18. The method of any one of claims 1-11, wherein the administration of the sorbent reduces the damage to the body caused by the hyperinflammatory response, thereby decreasing the patient's risk of immunoparalysis.

19. The method of any claims 1-11, wherein administration of the sorbent reduces T-cell or other immune cell exhaustion.

20. The method of any one of claims 1-11, wherein administration of the sorbent results in removal of one or more of a) cytokine and b) soluble ligand from bodily/physiologic fluid of the subject.

21. The method of any one of claims 1-11, wherein administration of the sorbent results in removal of inflammatory mediators.

22. The method of any one of claims 1-11, wherein administration of the sorbent results in removal of one or more checkpoint inhibitor proteins and/or their soluble counterparts.

23. The method of any one of claims 1-11, wherein administration of the sorbent results in partial or full restoration of T-cell or other immune cell function.

24. The method of claim 20, wherein the soluble ligand is one or more of sPD-L1, sPD-L2, sMICA, SULBP2, sFasL, sCTLA-4, MHC class II, and sCD40L.

25. The method of claim 20, wherein the cytokine comprises one or more of a member of interleukin, chemokine, interferon, lymphokine, tumor growth factor, or tumor necrosis factor family.

26. The method of any one of claims 1-25, wherein the sorbent comprises one or more residues of divinylbenzene and ethylvinylbenzene, styrene, and ethylstyrene monomers.

27. A method of reducing sepsis and inflammation related T-cell dysfunction in a subject comprising treating the subject with a therapeutically effective amount of porous biocompatible polymer sorbent.

28. The method of claim 27, wherein the sorbent comprises a range of pore diameters between about 50 Å to about 3000 Å and a pore volume between about 0.5 cc/g to about 3 cc/g dry polymer.

29. The method of claim 27, wherein the sorbent comprises a range of pore diameters between about 50 Å to about 40,000 Å and a pore volume between about 0.5 cc/g to about 5 cc/g dry polymer.

30. The method of any one of claims 27-29, wherein the sorbent is administered extracorporeally.

31. The method of any one of claims 27-29, wherein the sorbent is administered intravenously, intramuscularly, intratumorally, intradermally, intraperitoneally, subcutaneously, orally, rectally, topically, or nasally.

32. The method of claim 29, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

33. The method of any one of claims 27-32, wherein the sorbent is produced using at least one crosslinking agent and at least one monomer.

34. The method of claim 33, wherein the monomer comprises divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

35. The method of claim 33 or claim 34, wherein the crosslinking agent comprises one or more or divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

36. A method of treating immunoparalysis in a subject in need thereof comprising contacting a physiologic fluid of the subject with a porous biocompatible polymer sorbent, wherein method removes inflammatory mediators, and/or anti-inflammatory mediators, and/or checkpoint proteins.

37. The method of claim 36, wherein the sorbent comprises a range of pore diameters between about 50 Å to about 3000 Å and a pore volume between about 0.5 cc/g to about 3 cc/g dry polymer.

38. The method of claim 36, wherein the sorbent comprises a range of pore diameters between about 50 Å to about 40,000 Å and a pore volume between about 0.5 cc/g to about 5 cc/g dry polymer.

39. The method of any one of claims 36-38, wherein the contacting comprises administering the sorbent intravenously, intramuscularly, intratumorally, intradermally, intraperitoneally, subcutaneously, orally, rectally, topically, or nasally.

40. The method of any one of claims 36-38, wherein the contacting comprises administering the sorbent extracorporeally.

41. A method of treating immunoparalysis in a subject in need thereof comprising administering a porous biocompatible polymer sorbent to the subject,
wherein the administering places the sorbent in contact with a physiologic fluid of the subject.

42. The method of claim 41, wherein the sorbent comprises a range of pore diameters between about 50 Å to about 3000 Å and a pore volume between about 0.5 cc/g to about 3 cc/g dry polymer.

43. The method of claim 41, wherein the sorbent comprises a range of pore diameters between about 50 Å to about 40,000 Å and a pore volume between about 0.5 cc/g to about 5 cc/g dry polymer.

44. The method of any one of claims 41-43, wherein the physiologic fluid is blood.

45. The method of any one of claims 41-44, wherein the sorbent is administered extracorporeally.

46. The method of any one of claims 41-44, wherein the sorbent is administered intravenously, intramuscularly, intratumorally, intradermally, intraperitoneally, subcutaneously, orally, rectally, topically, or nasally.

47. A method of treating immunoparalysis in a subject in need thereof comprising providing to the subject a porous biocompatible polymer sorbent.

48. The method of claim 47, wherein the sorbent comprises a range of pore diameters between about 50 Å to about 3000 Å and a pore volume between about 0.5 cc/g to about 3 cc/g dry polymer.

49. The method of claim 47, wherein the sorbent comprises a range of pore diameters between about 50 Å to about 40,000 Å and a pore volume between about 0.5 cc/g to about 5 cc/g dry polymer.

50. The method of any one of claims 47-49, wherein the administration of the sorbent reduces the severity of immunoparalysis, immune suppression, or anergy.

51. The method of any one of claims 47-49, wherein the administration of the sorbent reduces the damage to the body caused by the hyperinflammatory response, thereby decreasing the patient's risk of immunoparalysis, immune suppression, or anergy.

52. The method of any one of claims 47-49, wherein administration of the sorbent reduces T-cell exhaustion.

53. The method of any one of claims 47-49, wherein the sorbent results in removal of one or more of a) cytokine and b) soluble ligand from bodily/physiologic fluid of the subject.

54. The method of any one of claims 47-49, wherein the sorbent results in removal of inflammatory mediators.

55. The method of any one of claims 47-49, wherein the sorbent results in removal of checkpoint inhibitor proteins.

56. The method of any one of claims 47-49, wherein the sorbent results in results in partial or full restoration of T-cell function.

57. The method of claim 53, wherein the soluble ligand is one or more of sPD-L1, sPD-L2, sMICA, SULBP2, sFasL, sCTLA-4, MHC class II, and sCD40L.

58. The method of claim 53, wherein the cytokine comprises one or more of a member of interleukin, chemokine, interferon, lymphokine, tumor growth factor, or tumor necrosis factor family.

59. The method of any one of claims 47-58, wherein the sorbent is administered extracorporeally.

60. The method of any one of claims 47-58, wherein the sorbent is administered intravenously, intramuscularly, intratumorally, intradermally, intraperitoneally, subcutaneously, orally, rectally, topically, or nasally.

61. A method of treating sepsis and/or inflammation related T-cell or immune cell dysfunction in a subject in need thereof comprising contacting a physiologic fluid of the subject with a porous biocompatible polymer sorbent.

62. The method of claim 61, wherein the sorbent comprises a range of pore diameters between about 50 Å to about 3000 Å and a pore volume between about 0.5 cc/g to about 3 cc/g dry polymer.

63. The method of claim 61, wherein the sorbent comprises a range of pore diameters between about 50 Å to about 40,000 Å and a pore volume between about 0.5 cc/g to about 5 cc/g dry polymer.

64. The method of any one of claims 61-63, wherein the contacting comprises administering the sorbent intravenously, intramuscularly, intratumorally, intradermally, intraperitoneally, subcutaneously, orally, rectally, topically, or nasally.

65. The method of any one of claims 61-63, wherein the contacting comprises administering the sorbent extracorporeally.

66. A method of treating sepsis and/or inflammation related T-cell or other immune cell dysfunction in a subject in need thereof comprising administering a porous biocompatible polymer sorbent to the subject,
wherein the administering places the sorbent in contact with a physiologic fluid of the subject.

67. The method of any one of claims 47-66, wherein the physiologic fluid is blood.

68. A method of treating sepsis and/or inflammation related T-cell dysfunction in a subject in need thereof comprising providing to the subject a porous biocompatible polymer sorbent.

69. The method of any one of claims 66-68, wherein the sorbent comprises a range of pore diameters between about 50 Å to about 3000 Å and a pore volume between about 0.5 cc/g to about 3 cc/g dry polymer.

70. The method of any one of claims 66-68, wherein the sorbent comprises a range of pore diameters between about 50 Å to about 40,000 Å and a pore volume between about 0.5 cc/g to about 5 cc/g dry polymer.

71. The method of any one of claims 66-68, wherein the contacting comprises administering the sorbent intravenously, intramuscularly, intratumorally, intradermally, intraperitoneally, subcutaneously, orally, rectally, topically, or nasally.

72. The method of any one of claims 66-68, wherein the contacting comprises administering the sorbent extracorporeally.

73. The method of claim 70, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

74. The method of any one of claims 66-73, wherein the sorbent is produced using at least one crosslinking agent and at least one monomer.

75. The method of claim 74, wherein the monomer comprises one or more of divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, and divinylformamide.

76. The method of claim 74 or claim 75, wherein the crosslinking agent comprises one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

77. The method of claim 74 or claim 75, wherein the sorbent is produced additionally utilizing one or both of at least one dispersing agent and at least one porogen.

78. The method of any one of claims 66-73, wherein the sorbent comprises a biocompatible and hemocompatible exterior coating that is covalently bound to the sorbent by free-radical grafting.

79. The method of any one of claims 66-73, wherein said sorbent is administered prior to administration of supportive therapies.

80. The method of any one of claims 66-73, wherein said sorbent is administered at the same time as administration of supportive therapies.

81. The method of any one of claims 66-73, wherein said sorbent is administered following administration of supportive therapies.

82. The method of claim 81, wherein the supportive therapy comprises administration of an immunotherapeutic agent selected from one or more of checkpoint inhibitors, monoclonal antibodies, and bi-specific T-cell engagers.

83. The method of any one of claims 66-73, wherein the sorbent comprises one or more residues of divinylbenzene and ethylvinylbenzene, styrene, and ethylstyrene monomers.

84. The method of any one of claims 1-26, wherein administration for the sorbent results in decreased endothelial sequestration of leukocytes and/or increased presence of leukocytes to the area of infection or injury, and/or reduction in infection and/or improved healing.
